(19) United States
(12) Patent Application Publication
(10) Pub. No.: US 2013/0216523 A1
(43) Pub. Date: Aug. 22, 2013

(54) Methods for Facilitating Diagnosis, Prognosis and Treatment of Cancer by Detecting HER1 Expression

(71) Applicants: Gerald J. Wallweber, Foster City, CA (US); John William Winslow, El Granada, CA (US)

(72) Inventors: Gerald J. Wallweber, Foster City, CA (US); John William Winslow, El Granada, CA (US)

(73) Assignee: LABORATORY CORPORATION OF AMERICA HOLDINGS, Burlington, NC (US)

(21) Appl. No.: 13/437,586

(22) Filed: Apr. 2, 2012

Related U.S. Application Data
(60) Provisional application No. 61/470,448, filed on Mar. 31, 2011.

(51) Int. Cl. G01N 33/574; A61P 35/00; A61K 31/517; A61K 39/395; G01N 33/577; A61K 31/5377

(52) U.S. Cl. 424/133.1; 435/7.21; 514/234.5; 514/266.4; 514/266.24; 424/142.1

(57) ABSTRACT

Methods are provided for facilitating the diagnosis of subjects with HER1-activated cancers. In addition, method of treating subjects with a cancer characterized as having high levels of activated HER1 are provided. Also provided are methods for determining or otherwise assessing the prognosis of an subject with a HER1-activated cancer. The methods include the analysis of samples for the presence or the absence of activated HER1 markers as indicated by HER1-HER1 homodimers, HER1 phosphorylation at position 1173, pan-phosphorylation of HER1 or associated molecules, or HER1-HER2 heterodimers. Activated HER1 measurements can be used to track a subject's response to a treatment regimen, predict the success of using a particular treatment regimen, determine the effects of a treatment regimen, or for categorizing a subject in order to create a homogenous group for a clinical trial.

### CROSS-REFERENCE TO RELATED APPLICATIONS

This application claims the benefit of and priority under 35 U.S.C. §119(e) to U.S. Provisional Application No. 61/470,448, filed Mar. 31, 2011, which is incorporated herein by reference in its entirety.

### FIELD OF THE INVENTION

The present invention relates generally to methods for analyzing HER1 expression in patients with a cancer. In particular, in some embodiments, various HER1 forms are detected to facilitate the diagnosis, prognosis, or targeted drug treatment of patients with a squamous cell carcinoma of the head and neck (SCCHN).

### BACKGROUND OF THE INVENTION

Certain members of the tyrosine kinase receptor superfamily that are associated with cell proliferation, survival, and migration are the human epidermal growth factor receptors or HER protein family. These HER proteins include HER1 (also known as EGFR and ErbB1), HER2 (ErbB2), HER3 (ErbB3), and HER4 (ErbB4). Expression levels of each of these individual cell surface receptors have been used as cancer biomarkers.

It is known that HER1 undergoes certain observable changes when activated. For example, upon binding to its ligand EGF, HER1 forms HER1-HER1 homodimers and/or HER1-HER2 heterodimers. These dimerized receptors undergo trans-phosphorylation, which activates downstream signaling pathways. HER1 becomes phosphorylated at multiple sites, including on the tyrosine residue at position 1173. In addition, the HER1 homodimers and heterodimers phosphorylate other substrates or other amino acid residues of the HER1 protein.

Increased HER1 expression has been observed in a wide variety of tumors, including non-small cell lung cancer (NSCLC), colorectal cancer (CRC), and SCCHN. The response rates of HER1 antibody monotherapy (13%) is comparable to the added response rates of HER1 antibody therapy when used in combination with chemotherapy (approximately 16%) in patients with recurrent and/or metastatic SCCHN (Vermorken et al., 2008, N. Engl. J. Med. 359:116; Vermorken et al., 2007, J. Clin. Oncol. 25:2171). Clinical studies have supported an important role of signaling through the EGFR in a subgroup of CRC cancers. Some studies have shown that advanced CRC with tumor-promoting mutation in signaling pathways downstream of EGFR activation (e.g., KRAS, BRAF, p110 subunit of PI3K) do not respond to anti-EGFR therapy. See Markowitz, S. D., et al., 2009, New Engl. J. Med. 361:2449-2460; Bardelli, A. and Siena, S., 2010, Clin. Oncology 28(7):1254-1261; Siena, S., et al., 2009, J. Natl. Cancer Inst. 101:1308-1324. In addition, despite its overexpression in nearly all NSCLC tumors, therapeutic inhibition of EGFR has resulted in significant tumor regressions in only 10-20% of patients. Jänne, P. A., et al., 2005, J. Clin. Oncology 23(14):3227-3234.

Thus, it has proven difficult to determine whether a subject with a cancer, particularly having SCCHN, NSCLC, or CRC, is likely or unlikely to respond to treatment with a HER1-acting agent. Conventional immunohistochemical (IHC) analysis, fluorescence in situ hybridization (FISH) analysis, and mRNA analysis have been used to detect HER1 expression to determine whether treatment with a HER1-acting agent, such as, e.g., cetuximab (ERBITUX™), is warranted. However, these standard measures of HER1 levels by IHC, mRNA detection, or gene copy number by FISH do not predict or correlate with the observed response rate to HER1 antibody monotherapy. Therefore, many subjects are being provided expensive treatment with a HER1-acting agent even though they are unlikely to respond to such treatment. Determining whether such patients are unlikely to respond to HER1-acting agents would avoid providing costly but ineffective treatment to those patients.

Because of the relatively low response rates and high treatment costs, alternative gene or protein expression signatures (biomarkers) are needed to identify patients mostly likely to respond to HER1 targeted therapies. What is needed, therefore, is a method to determine whether an individual will be responsive to HER1-acting agents so as to maximize the appropriate therapy for the individual. Also needed are compositions, systems, and kits for use in such methods.

### BRIEF SUMMARY OF THE INVENTION

The present invention provides methods for determining whether a subject with a cancer is likely to respond to treatment with a HER1-acting agent. In certain embodiments, the present invention provides methods to correlate the relative levels of the amount of an activated HER1 molecule in a biological sample from a subject having a cancer, with a prognosis for the likelihood that the subject will respond to treatment with a HER1 acting agent comprising: (a) detecting in a biological sample from the subject's cancer the amount of activated HER1; and (b) correlating the amount of activated HER1 to a prognosis for the likelihood that the subject will respond to treatment with a HER1 acting agent. In certain embodiments, the cancer is metastatic or recurrent squamous cell carcinoma of the head and neck (SCCHN). In some embodiments, the cancer is locogoregionally advanced SCCHN. In certain other embodiments, the cancer is non-small cell lung cancer (NSCLC). In certain embodiments, the cancer is colorectal cancer (CRC). In some embodiments, if the amount of the activated HER1 is equal to or above a threshold level, the subject's prognosis is to be likely to respond to the HER1 acting agent. In certain embodiments of the methods, if the amount of the activated HER1 is below a threshold level, the subject's prognosis is not to be likely to respond to the HER1 acting agent.

In certain embodiments, the activated HER1 is detected by determining the amount of a HER1-HER1 homodimer, HER1-HER2 heterodimer, phosphorylated HER1, or a combination thereof, that is present in the biological sample. In certain embodiments the activated HER1 is detected by binding of an activated HER1 binding compound attached to a detectable moiety. In one embodiment, the activated HER1 binding compound is one or more HER1 antibodies that bind to a HER1-HER1 homodimer, HER1-HER2 heterodimer, or phosphorylated HER1. In some embodiments, the amount of activated HER1 is detected by determining the amount of at least two HER1 molecules selected from the group consisting of a HER1-HER1 homodimer, HER1-HER2 heterodimer, and phosphorylated HER1 that is present in the biological sample. Phosphorylated HER1 in a biological sample may be detected, for example, by using a HER1 phosphospecific or pan antibody. In certain embodiments, the amount of phosphorylated HER1 in the biological sample is detected by using a phosphospecific antibody that binds HER1 protein that is phosphorylated at the tyrosine residue at position 1173 of HER1. In certain embodiments, the assay used for detecting activated HER1 is capable of measuring and/or quantifying an amount of protein-protein interactions in a sample.

In certain embodiments, total HER1 and activated HER1 are detected. In certain embodiments of the methods, the amount of both total HER1 and activated HER1 in the biological sample is detected. In some embodiments, a predetermined measure is created by dividing a plurality of subject samples into at least two subgroups, wherein the first subgroup comprises samples having total HER1 molecules at a low level in the biological sample, wherein the low level comprises having an amount of total HER1 molecules equal to or below a threshold level; and wherein the second subgroup comprises samples having total HER1 molecules at a high level, wherein the high level comprises having an amount of activated HER1 molecules above the threshold level. In other embodiments of the methods, a predetermined measure is generated by dividing the high level total HER1 subgroup into at least two subgroups based on the level of activated HER1 as determined by detecting the amount of HER1-HER1 homodimer, HER1-HER2 heterodimer, or phosphorylated HER1.

In certain other embodiments, the methods involve detecting the level of at least one other biomarker, wherein the biomarker is selected from the group consisting of HER2, HER3, phosphorylated-HER3, cMET, and a combination thereof.

In other aspects, the invention is drawn to a method for determining whether a subject with a cancer is likely to respond to treatment with a HER1-acting agent. In certain embodiments, the invention also provides methods for predicting a probability of a significant event in the time course of disease in a subject with a cancer based on the predicted sensitivity of a patient to a HER1-acting agent. In certain embodiments, the methods comprise detecting activated HER1 as described hereinafter, and determining whether the subject is likely to respond to treatment with the HER1-acting agent.

In certain embodiments, the HER1-acting agent comprises at least one agent selected from the group consisting of cetuximab, gefitinib, erlotinib, lapatinib, panitumumab, zalatumumab, nimotuzumab, and matuzumab.

In certain embodiments of each of the methods and/or aspects of the invention, the method comprises detecting in a biological sample from the subject's cancer the amount of activated HER1 and determining if the detected levels correlate to a low or high level of activated HER1 expression.

In certain aspects, methods are provided to facilitate the diagnosis of a squamous cell carcinoma of the head and neck (SCCHN) as compared to other cancer types of the head and neck. In some embodiments, the methods comprise: (a) detecting in a biological sample from the subject's cancer the amount of activated HER1; and (b) correlating the amount of activated HER1 to a diagnosis of SCCHN. In certain embodiments, if the amount of the activated HER1 is equal to or above a threshold level, the subject is diagnosed with SCCHN. In other embodiments, if the amount of the activated HER1 is below a threshold level, the subject is diagnosed as having a cancer type other than SCCHN.

Kits for use in correlating the relative levels of the amount of activated HER1 in a biological sample from a subject with a prognosis for the likelihood that the subject will respond to treatment with a HER1 acting agent are provided. The kits in certain embodiments comprise: (a) reagents for detecting in a biological sample from the subject's cancer the amount of activated HER1; and (b) instructions for correlating the amount of activated HER1 to a prognosis for the likelihood that the subject will respond to treatment with a HER1 acting agent.

Also provided are kits to facilitate the diagnosis of a squamous cell carcinoma of the head and neck (SCCHN) in a biological sample as compared to a different cancer type. In some embodiments, the kits comprise: (a) reagents for detecting in a biological sample from the subject's cancer the amount of activated HER1; and (b) instructions for correlating the amount of activated HER1 to a diagnosis of SCCHN.

### BRIEF DESCRIPTION OF THE DRAWINGS

The present invention may be better understood by reference to the following non-limiting figures.

FIG. 1 is a schematic diagram showing schematic representations of various VeraTag® assay formats in accordance with certain embodiments of the invention. Panels A and B show a light release format, while Panel C shows a reducing (DTT) format. Panel A depicts analysis of formalin-fixed paraffin-embedded (FFPE) tissue samples, and Panel B depicts analysis of a tissue lysate sample. In the light release format, diffusing reactive singlet oxygen may be used to cleave the covalent linker between a VeraTag® reporter molecule and a HER1 antibody in response to photo-induction of the cleavage-inducing agent by light. In the reducing format, a reducing agent is used to induce cleavage of the covalent linker between a VeraTag® reporter molecule and a HER1 antibody. Following cleavage, capillary electrophoretic (CE) separation of the VeraTag® reporter molecules may be conducted and assessed by electropherogram. The x-axis shows the time at which the cleaved VeraTag® reporter molecule eluted from the capillary, and the fluorescence intensity is shown on the y-axis. Fluorescent peaks v1 and v2 denote the elution of two different VeraTag® reporter molecules. “HER1” represents a HER1 monomer; “B” represents a biotin molecule; “S” represents a streptavidin molecule, “Tag” represents a VeraTag® reporter molecule, and “hv” represents light energy.

FIG. 2 provides information for each of the tumor samples studied. Panel A is a table showing the tissue source of the fifty-six SCCHN tumor samples and their total tissue distribution. Samples 7, 14, 15, 16, 17, 38, 56, 66, and 74 were determined to have high levels of expression of activated HER1 (boxed). Samples marked with an * or ̂ are from the same patient. Panel B is table showing the tissue source of the twenty colorectal samples that were studied. Samples 5, 8, and 18 in Panel B are samples were determined to have high levels of expression of activated HER1 (boxed). The KRAS mutation status is also identified for each CRC sample (WT: wild type KRAS gene; M: mutated KRAS gene (specific mutations identified listed for Panel B).

FIG. 3 is a series of graphs showing VeraTag® assay results for detection of certain biomarkers to determine consistency between batches of SCCHN samples assessed. Cell line control sample data are shown on the left side of each graph and CRC tumor sample data are shown on the right side of each graph. The fluorescence intensity for each sample is shown on the y-axis, measured in relative peak area (RPA). The solid dots indicate signal, and the open dots represent background. Panels A, B, C, and D show results from assays measuring levels of total HER1, HER2, HER3, and c-MET, respectively. Panels E, F, G, and H show results from assays measuring HER1-HER1 homodimers, HER1 phosphorylated at tyrosine 1173, pan phosphorylated HER1, and HER1-HER2 heterodimers, respectively. Measured signal are indicated by black circles () and background IgG signals are indicated by white circles (∘).

FIG. 4 is a series of graphs showing VeraTag® assay results for detection of certain biomarkers to determine consistency between batches of CRC samples assessed. Cell line control sample data are shown on the left side of each graph and CRC tumor sample data are shown on the right side of each graph. The fluorescence intensity for each sample is shown on the y-axis, measured in relative peak area (RPA). The fluorescence intensity for each sample is shown on the y-axis, measured in relative peak area (RPA). The biomarker levels measured are: HER1 (Panel A); HER2 (Panel B); HER3 (Panel C); HER1-HER1 homodimer (Panel D); and HER1pY1173 (Panel E). Measured signal are indicated by black circles () and background IgG signals are indicated by white circles (∘).

FIG. 5 provides data illustrating differences between squamous cell carcinoma of the oral mucosa (“SCC”) and other head and neck tumors (“Other”). Panels A-E are graphs showing biomarker levels as measured using the VeraTag® assay. The SCC samples are grouped on the right side of the graph, and the other head and neck tumor samples are grouped on the left side of the graph. Sample numbers are indicated on the x axis for Panels A to E, and RPA/TA is shown on the y-axis.

FIG. 6 shows box and whisker plots depicting the distribution of biomarker levels measured in FIGS. 5A-5E separated based on whether the sample was a SCC or Other sample. Panels A-E correspond to the data shown in Panels A-E of FIG. 5.

FIG. 7 shows a comparison of different biomarker measurement methods. Panels A-F are graphs comparing the results from the VeraTag® assays to the results from mRNA, FISH, and IHC experiments from the same SCCHN tumors. Panels A and B compare VeraTag® assay results to IHC results. Panel C compares VeraTag® results to FISH assay results. Panels D, E, and F compare results of VeraTag® assays for HER1, HER2, and HER3, respectively, with the corresponding mRNA results measured by deltaCt (change in cycle threshold), or the number of cycles necessary before the fluorescence reading surpasses the set baseline relative to an internal control.

FIG. 8 shows comparison of data generated by IHC analysis (Panel A) and VeraTag® assays (Panel B) for the CRC tumor samples. Horizontal lines represent the median measurement.

FIG. 9 shows the results of measuring HER1 levels in the SCCHN samples using multiple platforms. Horizontal lines represent the median measurement. Panels A and B show IHC data from two separate experiments. Panels C-E show data from VeraTag® assay, IHC, qPCR (mRNA), and FISH experiments. Tumor samples having high amounts of activated HER1 are indicated by white circles (∘).

FIG. 10 shows graphs illustrating the distribution of HER1 levels for the CRC tumors. The horizontal lines across each graph represent the median measurement. The left two graphs in Panel A show IHC data generated from two separate analyses of the samples, while the graph on the right shows data generated using the VeraTag® assay. Tumor samples having high amounts of activated HER1 are indicated by white circles (∘).

FIG. 11 shows pairwise comparison of the biomarker levels in the tumor samples. Biomarker levels were measured using VeraTag® assays. Panels A and B shows the data for the SCCHN tumor samples, and Panel C shows the data for the CRC samples. The Spearman correlation coefficients having significant p-values (p<0.05) are underlined.

FIG. 12 shows heat map diagrams illustrating biomarker levels for the tumor samples. Biomarker levels were measured using VeraTag® assays. Panel A is a heat map for each of the SCCHN tumor samples. Panel B is a heat map for each of the CRC samples. The sample number is indicated to the left of the heat map, and the biomarker analyzed in the assay is shown at the top of the heat map. Samples that exhibited the highest expression (>90^{th} percentile) are shown in black; and samples with low expression (<10^{th} percentile) are shown in grey. White is used to indicate samples with missing values. The samples identified with an arrow are the samples that were considered to have the highest levels of activated HER1.

FIG. 13 shows a hierarchical cluster analysis of the SCCHN tumor samples by biomarker level as measured by VeraTag® assays. The sample numbers are shown on the right of the figure. The biomarkers analyzed are shown at the bottom of the figure: HER1-HER2 heterodimers (H12D); HER1-HER1 homodimers (HID); HER1 phosphorylated at tyrosine 1173 (H1pY1173); pan-phosphorylated HER1 (H1pYPan); and total HER1 (H1T). JW: The tumors expressing the highest levels of activated HER1 or HER1 total expression are shown in black or dark grey, and identify 7/9 of the tumors expressing the highest levels activated HER1 seen in the heat map (FIG. 5).

FIG. 14 shows stratification of SCCHN samples based on analysis of two biomarkers. Biomarker levels were measured using VeraTag® assays (read out: relative peak area (RPA)). Tumors that are expected to have highly activated HER1 are depicted by (∘), while other samples are depicted by (). Panel A shows results analyzing HER1-HER1 homodimers (H1D) versus HER1 total (H1T) expression levels. Panel B shows results plotting the ratio of HER1-HER1 homodimers (H1D) to HER1 total expression (H1T) levels against HER1 total expression. Panel C shows results analyzing the ratio of HER1 phosphorylated at tyrosine 1173 (HER1pY1173) to HER1 total (HIT) versus HER1 total expression levels.

FIG. 15 shows stratification of CRC samples based on analysis of two biomarkers. Biomarker levels were measured using VeraTag® assays (read out: relative peak area (RPA)). Tumors that are expected to have highly activated HER1 are depicted by (∘), while other samples are depicted by (). Tumors that are highly activated HER1 tumors are depicted by (∘). Panel A shows results analyzing HER1-HER1 homodimers (HID) versus HER1 total (HIT) expression levels. Panel B shows results plotting the ratio of HER1-HER1 homodimers (HID) to HER1 total expression (HIT) levels against HER1 total expression. Panel C shows results analyzing the ratio of HER1 phosphorylated at tyrosine 1173 (H1pY1173) to HER1 total (HIT) versus HER1 total expression levels.

### DETAILED DESCRIPTION OF THE INVENTION

Embodiments of the present invention provide methods for facilitating the diagnosis and prognosis of cancer, and for assessing whether such cancer is likely to respond to treatment with a HER1-acting agent. The methods involve measuring the levels of the amount of an activated HER1 molecule in a biological sample from a subject having a cancer. In certain embodiments, the cancer is metastatic or recurrent squamous cell carcinoma of the head and neck (SCCHN), non-small cell lung cancer (NSCLC), or colorectal cancer (CRC). In some embodiments, the cancer is locoregionally advanced SCCHN. The amount of activated HER1 may be detected, for example, by determining the amount of a HER1-HER1 homodimer, HER1-HER2 heterodimer, phosphorylated HER1, or a combination thereof, that is present in the biological sample.

### DEFINITIONS AND ABBREVIATIONS

The term “about,” as used herein, unless otherwise indicated, refers to a value that is no more than 10% above or below the value being modified by the term. For example, the term “about 5 μg/kg” means a range of 4.5 μg/kg to 5.5 μg/kg. As another example, “about 1 hour” means a range of 48 minutes to 72 minutes.

As used herein, the term “activated HER1” refers to a molecular form of HER1 that is capable of initiating downstream signaling pathways. For example, activated HER1 may be in the form of a HER1 homodimer, a HER1-HER2 heterodimer, or a phosphorylated HER1. A phosphorylated HER1 may be phosphorylated at the tyrosine residue at position 1173, or at one or more of several additional tyrosine residues. In addition, activated HER1 may be detected by detecting the phosphorylation of other proteins that are associated with the activated HER1 protein.

As used herein, the term “antibody” means an immunoglobulin that specifically binds to, and is thereby defined as complementary with, a particular spatial and polar organization of another molecule. The antibody can be monoclonal, polyclonal, or recombinant and can be prepared by techniques that are well known in the art such as immunization of a host and collection of sera (polyclonal) or by preparing continuous hybrid cell lines and collecting the secreted protein (monoclonal), or by cloning and expressing nucleotide sequences or mutagenized versions thereof coding at least for the amino acid sequences required for specific binding of natural antibodies. Antibodies may include a complete immunoglobulin or fragment thereof, which immunoglobulins include the various classes and isotypes, such as IgA, IgD, IgE, IgG1, IgG2a, IgG2b and IgG3, IgM, etc. Fragments thereof may include Fab, Fv and F(ab′)₂, Fab′, and the like. Antibodies may also be single-chain antibodies or an antigen-binding fragment thereof, chimeric antibodies, humanized antibodies or any other antibody derivative known to one of skill in the art that retains binding activity that is specific for a particular binding site. In addition, aggregates, polymers, and conjugates of immunoglobulins or their fragments can be used where appropriate so long as binding affinity for a particular binding site is maintained. Guidance in the production and selection of antibodies and antibody derivatives for use in immunoassays, including such assays employing releasable molecular tag (as described below) can be found in readily available texts and manuals, e.g., Harlow and Lane, 1988, Antibodies: A Laboratory Manual, Cold Spring Harbor Laboratory Press, New York; Howard and Bethell, 2001, Basic Methods in Antibody Production and Characterization, CRC Press; Wild, ed., 1994, The Immunoassay Handbook, Stockton Press, New York.

“Antibody binding composition” is used herein to refer to a molecule or a complex of molecules that comprises one or more antibodies, or antigen-binding fragments that bind to a molecule, and derives its binding specificity from such antibody or antibody-binding fragment. Antibody binding compositions include, but are not limited to, (i) antibody pairs in which a first antibody binds specifically to a target molecule and a second antibody binds specifically to a constant region of the first antibody; a biotinylated antibody that binds specifically to a target molecule and a streptavidin protein, which protein is derivatized with moieties such as molecular tags or photosensitizers or the like, via a biotin moiety; (ii) antibodies specific for a target molecule and conjugated to a polymer, such as dextran, which, in turn, is derivatized with moieties such as molecular tags or photosensitizers, either directly by covalent bonds or indirectly via streptavidin-biotin linkages; (iii) antibodies specific for a target molecule and conjugated to a bead, or microbead, or other solid phase support, which, in turn, is derivatized either directly or indirectly with moieties such as molecular tags or photosensitizers, or polymers containing the latter.

“Antigenic determinant” or “epitope” are used interchangeably herein to refer to a site on the surface of a molecule, usually a protein, to which a single antibody molecule binds. Generally, a protein has several or many different antigenic determinants and reacts with antibodies of different specificities. One preferred antigenic determinant is a phosphorylation site of a protein.

As used herein, the terms “aspect” and “embodiment” are used interchangeably.

“Binding compound” shall refer herein to an antibody binding composition, an antibody, a peptide, a peptide or non-peptide ligand for a cell surface receptor, a protein, an oligonucleotide, an oligonucleotide analog, such as a peptide nucleic acid, a lectin, or any other molecular entity that is capable of specifically binding to a target protein or molecule or stable complex formation with an analyte of interest, such as a complex of proteins. In one aspect, a binding compound, which can be represented by the formula below, comprises one or more molecular tags attached to a binding moiety.

“Binding moiety” refers to any molecule to which molecular tags can be directly or indirectly attached that is capable of specifically binding to an analyte. Binding moieties include, but are not limited to, antibodies, antibody binding compositions, peptides, proteins, nucleic acids, and organic molecules having a molecular weight of up to about 1000 daltons and containing atoms selected from the group consisting of hydrogen, carbon, oxygen, nitrogen, sulfur and phosphorus. Preferably, binding moieties are antibodies or antibody binding compositions.

As used herein, “cancer” and “cancerous” refer to or describe the physiological condition organism, including mammals, that is typically characterized by unregulated cell growth. Examples of cancer include, but are not limited to, carcinoma, lymphoma, blastoma, sarcoma and leukemia. More particular examples of such cancers include squamous cell carcinoma, lung cancer, e.g., small-cell lung cancer or non-small cell lung cancer; gastrointestinal cancer, pancreatic cancer, glioblastoma, cervical cancer, ovarian cancer, liver cancer, bladder cancer, hepatoma, breast cancer, colon cancer, colorectal cancer, endometrial carcinoma, salivary gland carcinoma, kidney cancer, prostate cancer, vulval cancer, thyroid cancer, hepatic carcinoma and various types of head and neck cancer.

“Chemotherapeutic agent” means a chemical substance, primarily a cytotoxic or cytostatic agent, that is used to treat a condition, particularly cancer. Chemotherapeutic agents shall include such compounds as paclitaxel, as set forth herein.

As used herein, the phrase “cleavable linkage” refers to a chemical linking group that may be cleaved under conditions that do not degrade the structure or affect detection characteristics of a molecular tag connected to a binding moiety with the cleavable linkage.

“Cleavage-inducing moiety” or “cleaving agent” are used interchangeably herein to refer to a group that produces an active species that is capable of cleaving a cleavable linkage, for example, by oxidation. Preferably, the active species is a chemical species that exhibits short-lived activity so that its cleavage-inducing effects are only in the proximity of the site of its generation.

A “cleaving probe,” as used herein, refers to a reagent that comprises a cleavage-inducing moiety as defined herein and an antibody binding composition, an antibody, a peptide, a peptide or non-peptide ligand for a cell surface receptor, a protein, such as biotin or streptavidin, an oligonucleotide, an oligonucleotide analog, such as a peptide nucleic acid, a lectin or any other molecular entity that is capable of specifically binding to a target protein or molecule or stable complex formation with an analyte of interest, such as a complex of proteins.

“FFPE” or “formalin-fixed, paraffin-embedded” refer to a group of cells or quantity of tissue that are fixed, particularly conventional formalin-fixed, paraffin-embedded samples. Such samples are typically, without limitation, used in an assay for receptor complexes in the form of thin sections, e.g. 3-10 μm thick, of fixed tissue mounted on a microscope slide or equivalent surface. Such samples also typically undergo a conventional re-hydration procedure, and optionally, an antigen retrieval procedure as a part of, or preliminary to, assay measurements. In some embodiments, the sections are about 5 μm thick sections of fixed tissue cut onto positively charged glass sides.

As used herein, “greater than or equal to” (i.e., ≧ or >=) can in certain alternative embodiments mean “greater than” (>). Also, as used herein, “less than or equal to” (i.e., ≦ or <=) can in certain alternative embodiments mean “less than” (<).

“Hazard ratio”, as used herein, refers to a statistical method used to generate an estimate for relative risk. “Hazard ratio” is the ratio between the predicted hazard of one group versus another group. For example, patient populations treated with, versus without, a HER1-acting agent can be assessed for whether or not the HER1-acting agent is effective in increasing the time to distant recurrence of disease. The hazards ratio can then be compared to an independent measure, such as the ratio of activated HER1 to total HER1. At activated HER1 to total HER1 ratios at which the hazards ratio is less than one, treating with a HER1-acting agent has a greater chance of efficacy. At activated HER1 to total HER1 ratios at which the hazards ratio is indistinguishable from one, treating with a HER1 acting agent has a lower chance of efficacy.

“HER1,” “Her1,” “Her-1,” “EGFR,” “ErbB1,” and the like are used interchangeably herein to refer to native HER1, and allelic variants thereof, as described, for example, in Cohen et al., 1980, J. Biol. Chem. 255:4834-42, and GenBank Accession No. NM 005228 (see http://www.ncbi.nlm.nih.gov/nuccore/NM_{—}005228). Unless indicated otherwise, the terms “HER1,” “Her1,” “Her-1,” “EGFR,” “ErbB1,” and the like, when used herein, refer to the human protein. The gene encoding HER1 is referred to herein as “erbB1.” As used herein, H1T shall refer to total HER1 expression.

“HER1-acting agent” or “HER1-targeted therapy”, as used herein, refers to a compound that can inhibit a biological activity of HER1 or a HER1 expressing cell or a HER1 positive cancer cell. Such biological activities include, but are not limited to, dimerization, autophosphorylation, phosphorylation of another receptor, signal transduction and the like. Biological activities can include, without limitation, cell survival and cell proliferation, and inhibition of such activities by a HER1-acting agent could be direct or indirect cell killing (e.g., ADCC), disruption of protein complexes or complex formation, modulation of protein trafficking or enzyme inhibition. Biological activities can also include patient response as set forth in this application. It will be appreciated that, as used herein, a “HER1-acting agent” encompasses molecules capable of interfering with, blocking, reducing or modulating the interaction between EGF receptor (EGFR) and EGF (HER1), or other ligand capable of binding to EGFR. Exemplary HER1-acting agents include, but are not limited to, cetuximab (ERBITUX™), gefitinib (IRESSA™), erlotinib (TARCEVA™), lapatinib (TYKERB™), panitumumab (VECTIBIX™), zalatumumab (GenMab Corp.), nimotuzumab (YM Biosciences & TheraCim), and matuzumab (Merck/Serono).

The phrase “HER1 homodimer” in reference to cell surface HER1 membrane receptors means a complex of two or more membrane-bound HER1 proteins. Dimers usually consist of two receptors in contact with one another. Dimers may be created in a cell surface membrane by passive processes, such as Van der Waal interactions, and the like, or dimers may be created by active processes, such as by ligand-induced dimerization, covalent linkages, interaction with intracellular components or the like. See, e.g., Schlessinger, 2000, Cell 103:211-225. As used herein, the term “dimer” is understood to refer to “cell surface membrane receptor dimer,” unless understood otherwise from the context.

The phrase “activated HER1 to total HER1 ratio” refers to a measure that describes the amount of activated HER1 molecules divided by the total amount of HER1 in a sample from a subject's tissue according to any single quantitative method available to one skilled in the art.

A “HER1 positive” or “HER1-activated” cancer, cancer cell, subject or patient, as used herein, refers to a cancer cell, subject, or patient, that has elevated levels of activated HER1.

“Her-2”, “ErbB2”, “c-Erb-B2”, “HER2”, “Her2” and “neu” are used interchangeably herein and refer to native Her-2, and allelic variants thereof, as described, for example, in Semba et al., 1985, Proc. Nat. Acad. Sci. USA 82:6497-650 and Yamamoto et al., 1986, Nature 319:230-234 and Genebank accession number X03363. Unless indicated otherwise, the terms “Her-2”, “ErbB2”, “c-Erb-B2”, “HER2” and “Her2” when used herein refer to the human protein. The gene encoding Her2 is referred to herein as “erbB2.” As used herein, H2T shall refer to total Her-2 expression.

“High” refers to a measure that is greater than normal, greater than a standard such as a predetermined measure or a subgroup measure or that is relatively greater than another subgroup measure. For example, high total HER1 refers to a measure of HER1 that is greater than a normal HER1 measure. A normal HER1 measure may be determined according to any method available to one skilled in the art. High HER1 may also refer to a measure that is equal to or greater than a predetermined measure, such as a predetermined cutoff. High HER1 may also refer to a measure of HER1 wherein a high HER1 subgroup has relatively greater levels of HER1 than another subgroup. For example, without limitation, according to the present specification, two distinct patient subgroups can be created by dividing samples around a mathematically determined point, such as, without limitation, a median, thus creating a subgroup whose measure is high (i.e., higher than the median) and another subgroup whose measure is low. HER1 can be measured by any method known to one skilled in the art such as, for example, without limitation, using VeraTag® or using any standard immunohistochemical (IHC) method.

As another example, high activated HER1 refers to a measure of activated HER1 that is greater than a normal measure of activated HER1 in a particular set of samples or patients that are HER1 positive. A normal activated HER1 measure may be determined according to any method available to one skilled in the art. High activated HER1 may also refer to a measure that is greater than a predetermined measure, such as a predetermined cutoff. High activated HER1 may also refer to a measure of activated HER1 wherein a high activated HER1 subgroup has a relatively higher level of activated HER1 than another subgroup. Activated HER1 can be measured by any method known in the art such as Fluorescence resonance energy transfer (FRET), Bioluminescent resonance energy transfer (BRET), proximity ligation assay (PLA), dimer-specific antibodies or VeraTag® or any other method that is well known to one skilled in the art.

As another example, high activated HER1 to total HER1 ratio may refer to the one or more subgroups of activated HER1 to total HER1 ratios that have measures greater than low ratio subgroups. High activated HER1 to total HER1 ratios may be determined according to any individual quantitative methods available to one skilled in the art. In some cases, a “high” expression level may comprise a range of expression that is very high and a range of expression that is “moderately high” where moderately high is a level of expression that is greater than normal, but less than “very high”. Example ranges for high (including very high and moderately high) Her-2 expression are provided herein.

“Moderately high” “medium” or “intermediate”, as used herein, refers to a measure that is greater than “low” and less than very “high”. For example, “intermediate” may be used to describe one or more of the at least 3 subgroups that fall in the middle range of measures of activated HER1 to total HER1 ratios.

“Likely to,” as used herein, refers to an increased probability that an item, object, thing or person will occur. Thus, in one example, a subject that is likely to respond to treatment with a HER1-acting agent, such as cetuximab, has an increased probability of responding to treatment with the HER1-acting agent relative to a reference subject or group of subjects.

“Long,” as used herein, refers to a time measure that is greater than normal, greater than a standard such as a predetermined measure or a subgroup measure that is relatively longer than another subgroup measure. For example, with respect to a patient's longevity, a long time progression refers to time progression that is longer than a normal time progression. Whether a time progression is long or not may be determined according to any method available to one skilled in the art. Long could include, for example, no progression. In one embodiment, “long” refers to a time that is greater than the median time course required for a significant event to occur in a disease.

“Low” is a term that refers to a measure that is less than normal, less than a standard such as a predetermined measure, or a subgroup measure that is relatively less than another subgroup measure. For example, low HER1 means a measure of HER1 that is less than a normal HER1 measure in a particular set of samples of patients that is HER1 positive. A normal HER1 measure may be determined according to any method available to one skilled in the art. Low HER1 may also mean a method that is less than a predetermined measure, such as a predetermined cutoff. Low HER1 may also mean a measure wherein a low HER1 subgroup is relatively lower than another subgroup. For example, without limitation, according to the present specification, two distinct patient subgroups can be created by dividing samples around a mathematically determined point, such as, without limitation, a median, thus creating a group whose measure is low (i.e., less than the median) with respect to another group whose measure is high. HER1 can be measured by any method known to one skilled in the art such as, for example, without limitation, using a VeraTag® assay or using any standard immunohistochemical (IHC) method.

As another example, low activated HER1 means a measure of activated HER1 that is less than a normal measure of activated HER1 in a particular set of samples or patients that is HER1 positive. Low activated HER1 may also mean a measure that is less than a predetermined measure, such as a predetermined cutoff. Low activated HER1 may also mean a measure wherein a low activated HER1 subgroup is relatively less than another subgroup. Activated HER1 homodimers can be measured by any method known in the art such as Fluorescence resonance energy transfer (FRET), Bioluminescent resonance energy transfer (BRET), proximity ligation assay (PLA), dimer-specific antibodies or VeraTag® or any other method that is well known to one skilled in the art. As another example, low activated HER1 to total HER1 ratio may refer to the one or more subgroups of activated HER1 to total HER1 ratios that have measures less than either intermediate or high ratio subgroups. Low activated HER1 to total HER1 ratios may be determined according to any individual quantitative method available to one skilled in the art. Example ranges for low values of HER1 expression are provided herein.

A “molecular tag,” as used herein, refers to a molecule that can be distinguished from other molecules based on one or more physical, chemical or optical differences among the molecules being separated, including but not limited to, electrophoretic mobility, molecular weight, shape, solubility, pKa, hydrophobicity, charge, charge/mass ratio, polarity or the like. In one aspect, molecular tags in a plurality or set differ in electrophoretic mobility and optical detection characteristics and can be separated by electrophoresis. In another aspect, molecular tags in a plurality or set may differ in molecular weight, shape, solubility, pKa, hydrophobicity, charge, polarity and can be separated by normal phase or reverse phase HPLC, ion exchange HPLC, capillary electrochromatography, mass spectroscopy, gas phase chromatography or like technique. As described herein, a VeraTag® reporter molecule is a type of molecular tag.

“Optimal cutoff as used herein, refers to the value of a predetermined measure on subjects exhibiting certain attributes that allow the best discrimination between two categories of an attribute. For example, finding a value for an optimal cutoff that allows one to best discriminate between two categories, high H1T expression and low H1T expression, for determining overall survival. Optimal cutoffs are used to separate the subjects with values lower than or higher than the optimal cutoff to optimize the prediction model, for example, without limitation, to maximize the specificity of the model, maximize the sensitivity of the model, maximize the difference in outcome, or minimize the p-value from hazard ratio or a difference in response.

“Overall survival” or “OS” refers to a time as measured from the start of treatment to death or censor. Censoring may come from a study end or change in treatment. Overall survival can refer to a probability as, for example, a probability when represented in a Kaplan-Meier plot of being alive at a particular time, that time being the time between the start of the treatment to death or censor.

“Photosensitizer” shall mean a light-adsorbing molecule that when activated by light converts molecular oxygen into singlet oxygen. As used herein, “dithiothreitol” or “DTT” may be used as a substitute for a photosensitizer to cleave the VeraTag® reporter molecule by reduction.

“RECIST” shall mean an acronym that stands for “Response Evaluation Criteria in Solid Tumours” and is a set of published rules that define when cancer patients improve (“respond”), stay the same (“stable”) or worsen (“progression”) during treatments. Response as defined by RECIST criteria have been published, for example, at Journal of the National Cancer Institute, Vol. 92, No. 3, Feb. 2, 2000 and RECIST criteria may include other similar published definitions and rule sets. One skilled in the art would understand definitions that go with RECIST criteria, as used herein, such as “PR,” “CR,” “SD” and “PD.”

“Relative peak area” or “RPA” are used interchangeably and shall refer to the ratio between the fluorescence measurement of a particular VeraTag® reporter molecule and the fluorescence measurement of a known internal fluorescence standard of known and constant concentration

“Responsiveness,” to “respond” to treatment, and other forms of this verb, as used herein, refer to the reaction of a subject to treatment with a Her-2-acting agent. As an example, a subject responds to treatment with a Her2-acting agent if growth of a tumor in the subject is retarded about 10%, 20%, 30%, 40%, 50%, 60%, 70%, 80%, 90% or more. In another example, a subject responds to treatment with a Her-2-acting agent if a tumor in the subject shrinks by about 5%, 10%, 20%, 30%, 40%, 50% or more as determined by any appropriate measure, e.g., by mass or volume. In another example, a subject responds to treatment with a Her2-acting agent if the subject experiences a life expectancy extended by about 5%, 10%, 20%, 30%, 40%, 50% or more beyond the life expectancy predicted if no treatment is administered. In another example, a subject responds to treatment with a Her-2-acting agent if the subject has an increased disease-free survival, overall survival or increased time to progression. Several methods may be used to determine if a patient responds to a treatment including the RECIST criteria, as set forth above.

The terms “sample,” “tissue sample,” “patient sample,” “patient cell or tissue sample,” or “specimen” each refer to a collection of similar cells obtained from a tissue of a subject or patient. The source of the tissue sample may be solid tissue as from a fresh, frozen, and/or preserved organ or tissue sample or biopsy or aspirate; blood or any blood constituents; bodily fluids such as cerebral spinal fluid, amniotic fluid, peritoneal fluid or interstitial fluid; or cells from any time in gestation or development of the subject. The tissue sample may contain compounds that are not naturally intermixed with the tissue in nature such as preservatives, anticoagulants, buffers, fixatives, nutrients, antibiotics or the like. Cells may be fixed in a conventional manner (e.g., formalin-fixed, paraffin-embedding (FFPE)).

“Short,” as used herein, refers to a time measure that is shorter than normal, shorter than a standard such as a predetermined measure or a subgroup measure that is relatively shorter than another subgroup measure. For example, with respect to a patient's longevity, a short time progression refers to time progression that is shorter than a normal time progression. Whether a time progression is short or not may be determined according to any method available to one skilled in the art. In one embodiment, “short” refers to a time that is less than the median time course required for a significant event to occur in a disease.

As used herein, “significant event” shall refer to an event in a patient's disease that is important as determined by one skilled in the art. Examples of significant events include, for example, without limitation, primary diagnosis, death, recurrence, the determination that a patient's disease is metastatic, relapse of a patient's disease or the progression of a patient's disease from any one of the above noted stages to another. A significant event may be any important event used to assess OS, TTP and/or using the RECIST or other response criteria, as determined by one skilled in the art.

As used herein, the terms “subject” and “patient” are used interchangeably. As used herein, the terms “subject” and “subjects” refer to an animal, preferably a mammal including a non-primate (e.g., a cow, pig, horse, donkey, goat, camel, cat, dog, guinea pig, rat, mouse, sheep) and a primate (e.g., a monkey, such as a cynomolgous monkey, gorilla, chimpanzee and a human).

As used herein, “time course” shall refer to the amount of time between an initial event and a subsequent event. For example, with respect to a patient's cancer, time course may relate to a patient's disease and may be measured by gauging significant events in the course of the disease, wherein the first event may be diagnosis and the subsequent event may be metastasis, for example.

“Time to progression” or “TTP” refers to a time as measured from the start of the treatment to progression or a cancer or censor. Censoring may come from a study end or from a change in treatment. Time to progression can also be represented as a probability as, for example, in a Kaplan-Meier plot where time to progression may represent the probability of being progression free over a particular time, that time being the time between the start of the treatment to progression or censor.

“Treat,” “treatment,” and other forms of this word refer to the administration of a Her-2-acting agent to impede growth of a cancer, to cause a cancer to shrink by weight or volume, to extend the expected survival time of the subject and or time to progression of the tumor or the like.

“Unlikely to” refers to a decreased probability that an event, item, object, thing or person will occur with respect to a reference. Thus, a subject that is unlikely to respond to treatment with a HER1-acting agent has a decreased probability of responding to treatment with a HER1-acting agent relative to a reference subject or group of subjects.

“VeraTag®” and “VeraTag® assay” are used interchangeably herein and refer to single and multiplexed immunoassays, both single- and multi-label format, and the materials, methods and techniques for performing and utilizing such assays, including but not limited to reagents, analytical procedures and software related to those assays. Labels in the context of a VeraTag® assay are detectable moieties that are referred to as VeraTag® reporter molecules. Such assays are disclosed in this application as well as in U.S. Pat. No. 7,648,828 and U.S. Patent Application Nos. 2010/0143927; 2010/0233732; and 2010/0210034, which are incorporated by reference herein in their entireties.

As used herein, “VeraTag® reporter molecule” or “vTag,” are used to refer to a detectable moiety that is attached to an antibody used in a VeraTag® assay.

### Methods Involving Analysis of HER1

Embodiments of the invention provide methods for facilitating the diagnosis or prognosis of an individual by analyzing activated HER1 expression in a biological sample from the individual. In certain other embodiments, the methods involve determining whether a subject with a cancer is likely to respond to treatment with a HER1-acting agent and/or for predicting a time course of disease and/or a probability of a significant event in the time course of disease in a subject with a cancer. In certain embodiments, the method comprises detecting an activated HER1 biomarker or combination of biomarkers associated with responsiveness to treatment with a HER1-acting agent as described herein, and determining whether the subject is likely to respond to treatment with the HER1-acting agent. In certain embodiments, the methods comprise detecting a HER1 biomarker or combination of biomarkers and predicting a time course associated with progression of disease or a probability of a significant event in the time course of disease in a subject with cancer.

In some embodiments, the invention is drawn to a method for determining whether a subject with a cancer is likely to respond to treatment with a HER1-acting agent. In certain embodiments, the invention comprises a method of identifying a subpopulation of patients having a HER1-activated cancer that is likely to respond to a HER1-targeted therapy. In certain embodiments, the invention is drawn to a method for predicting a time course of disease. In some embodiments, the method is drawn to a method for predicting a probability of a significant event in the time course of the disease. In certain embodiments, the invention is drawn to a method for facilitating diagnosis of a HER1-activated cancer. In certain embodiments, the invention is drawn to a method for differentiating the histological subtype of a head and neck cancer. In certain embodiments, a time course is measured by determining the time between significant events in the course of a patient's disease, wherein the measurement is predictive of whether a patient has a long time course. For example, in some embodiment, the significant event is the progression from primary diagnosis to death. In some embodiments, the significant event is the progression from primary diagnosis to metastatic disease. In some embodiments, the significant event is the progression from primary diagnosis to relapse. In certain embodiments, the significant event is the progression from metastatic disease to death. In some embodiments, the significant event is the progression from metastatic disease to relapse. In certain embodiments, the significant event is the progression from relapse to death. In certain embodiments, the time course is measured with respect to overall survival rate, time to progression and/or using the RECIST or other response criteria. In some embodiments, the HER1-acting agent comprises at least one agent selected from the group consisting of cetuximab, gefitinib, erlotinib, lapatinib, panitumumab, zalatumumab, nimotuzumab, and matuzumab.

In some embodiments, the invention comprises a method for predicting responsiveness of a subject having a cancer to a HER1 acting agent comprising: (a) measuring the amount of activated HER1 in a biological sample from the subject's cancer; (b) determining whether the amount of activated HER1 in the subject's sample is above or below a threshold level; and (c) indicating that the subject is more likely to respond to the HER1 acting agent if the amount of activated HER1 in the subject's sample is equal to or above the threshold level than if the amount of activated HER1 is below the threshold level. In certain embodiments, responsiveness to a HER1-acting agent comprises a longer disease time course between diagnosis and the occurrence of a significant event while the subject is being treated with a HER1-acting agent. In some embodiments, the significant event comprises at least one of progression of the cancer from one stage to a more advanced stage, progression to metastatic disease, relapse, surgery, or death. In some embodiments, the HER1-acting agent comprises at least one agent selected from the group consisting of cetuximab, gefitinib, erlotinib, lapatinib, panitumumab, zalatumumab, nimotuzumab, and matuzumab.

In some embodiments, the invention comprises a method of identifying a subpopulation of subjects having a HER1-activated cancer that is likely to respond to a HER1-targeted therapy comprising (a) measuring an amount of activated HER1 in biological samples of cancer from a population of subjects with cancer; (b) determining whether the amount of activated HER1 in each subject's sample is above or below a threshold level; and (c) indicating whether each subject is more likely to respond to the HER1 acting agent based on the amount of activated HER1 in the biological sample from each subject, wherein if the amount of activated HER1 in the subject's sample is equal to or above the threshold level the subject is more likely to respond to a HER1-targeted therapy than if the amount of activated HER1 is below the threshold level, and (d) identifying the subjects that are more likely to respond to a HER1-targeted therapy as part of the subpopulation. In some embodiments, the HER1-acting agent comprises at least one agent selected from the group consisting of cetuximab, gefitinib, erlotinib, lapatinib, panitumumab, zalatumumab, nimotuzumab, and matuzumab. In certain embodiments, the identified subpopulation of subjects is indicated as appropriate for a clinical trial for a HER1-targeted therapy.

In certain embodiments, the invention comprises a method of diagnosing a squamous cell carcinoma of the oral mucosa in a subject having a cancer, comprising: (a) measuring an amount of activated HER1 in a biological sample from the subject's cancer; (b) determining whether the amount of activated HER1 in the subject's sample is above or below a threshold level; and (c) indicating that the subject likely to have a squamous cell carcinoma of the oral mucosa if the amount of activated HER1 in the subject's sample is equal to or above the threshold level as compared to if the amount of activated HER1 is below the threshold level.

In some embodiments, the invention comprises a method of determining if a subject having a cancer has high levels of activated HER1, comprising: (a) measuring the amount of activated HER1 in a biological sample from the subject's cancer; and (b) determining whether the amount of activated HER1 in the subject's sample is above or below a threshold level; and (c) indicating that the subject's cancer has a high level of activated HER1 if the amount of activated HER1 in the biological sample is equal to or above the threshold level. In certain embodiments, the subject is identified as appropriate to receive administration of a particular treatment regimen based on having a high amount of activated HER1. In some embodiments, the subject is identified as appropriate to receive administration of a particular treatment regimen based on having a low amount of activated HER1.

In certain embodiments, the invention comprises a method of determining if a subject having a head and neck cancer has squamous cell carcinoma of the head and neck (SCCHN), comprising: (a) measuring the amount of activated HER1 in a biological sample from a subject having a head and neck cancer; (b) determining whether the amount of activated HER1 in the subject's sample is above or below a threshold level; and (c) indicating that the subject has SCCHN if the amount of activated HER1 in the subject's sample is equal to or greater than the threshold level.

In some embodiments, the invention comprises a method of treating a subject with a cancer characterized as having high levels of activated HER1 comprising administering a HER1-targeted therapy to the subject. In certain embodiments, the cancer is at least one of squamous cell carcinoma of the head and neck (SCCHN), colorectal cancer, or lung cancer. In some embodiments, the HER1-acting agent comprises at least one agent selected from the group consisting of cetuximab, gefitinib, erlotinib, lapatinib, panitumumab, zalatumumab, nimotuzumab, and matuzumab. In some embodiments, the invention further comprises identifying whether the subject's cancer has high levels of activated HER1. In certain embodiments, the method of identifying whether the subject's cancer has a high level of activated HER1 comprises: (a) measuring the amount of activated HER1 in a biological sample from the subject's cancer; (b) determining whether the amount of activated HER1 in the subject's sample is above or below a threshold level; and (c) indicating that the subject's cancer has a high level of activated HER1 if the amount of activated HER1 in the subject's sample is equal to or above the threshold level.

Additional embodiments relating to the above embodiments and aspects are described below.

In certain embodiments, the method comprises detecting in a biological sample from the subject's cancer the amount of total HER1 and/or activated HER1 wherein if the amount of total HER1 and activated HER1 is high, then the patient is likely to respond to the HER1-acting agent and/or the patient has a long time course.

Thus, in certain embodiments, the invention comprises methods to correlate the relative levels of the amount of activated HER1 in a biological sample from a subject with a prognosis for the likelihood that the subject will respond to treatment with a HER1-acting agent comprising: (a) detecting in a biological sample from the subject's cancer the amount of activated HER1; and (b) correlating the amount of activated HER1 to a prognosis for the likelihood that the subject will respond to treatment with a HER1-acting agent.

In certain embodiments, if the amount of the activated HER1 is equal to or above a first threshold level, the subject's prognosis is to be likely to respond to the HER1-acting agent. Alternatively, if the amount of the activated HER1 is lower than the threshold level, the subject's prognosis is to be unlikely to respond to the HER1-acting agent

Also, in certain embodiments, a predetermined measure is created by dividing a plurality of subject samples into at least two subgroups, wherein the first subgroup comprises samples having total HER1 molecules at a low level in the biological sample, wherein the low level comprises having an amount of total HER1 molecules equal to or below a threshold level; and wherein the second subgroup comprises samples having total HER1 molecules at a high level, wherein the high level comprises having an amount of activated HER1 molecules above the threshold level. In other embodiments of the methods, a predetermined measure is generated by dividing the high level total HER1 subgroup into at least two subgroups based on the level of activated HER1 as determined by detecting the amount of HER1 homodimer, HER1/HER2 heterodimer, or phosphorylated HER1.

As discussed herein, in certain embodiments, the amount of activated HER1 is measured using an assay capable of measuring and/or quantifying an amount of protein-protein interactions in a sample.

In certain embodiments, the cancer is SCCHN, NSCLC, or CRC. In one embodiment, the cancer is SCCHN. In some embodiments, the cancer is metastatic or recurrent. In some embodiments, the cancer is early stage cancer. Alternatively, any cancer that may be sensitive to a HER1-acting agent may be analyzed or monitored. The HER1-acting agent may be any HER1-acting agent. In certain embodiments, the HER1-acting agent is one of the agents described herein. For example, in certain embodiments, the HER1-acting agent is cetuximab.

In certain embodiments of each of the methods of the invention, the samples may be analyzed for at least one other biomarker. For example, in some embodiments, the at least one other biomarker is selected from the group consisting of HER2, HER3, and cMET. In other embodiments, the at least one other biomarker is selected from the group consisting of FOXM1, PRAME, Bc12, STK15, CEGP1, Ki-67, GSTM1, CA9, PR, BBC3, NME1, SURV, GATA3, TFRC, YB-1, DPYD, GSTM3, RPS6 KB1, Src, Chk1, ID1, EstR1, p27, CCNB1, XIAP, Chk2, CDC25B, IGF1R, AK055699, PI3KC2A, TGFB3, BAGI1, CYP3A4, EpCAM, VEGFC, pS2, hENT1, WISP1, HNF3A, NFKBp65, BRCA2, EGFR, TK1, VDR, Contig51037, pENT1, EPHX1, IF1A, CDH1, HIF1a, IGFBP3; CTSB, DIABLO, VEGF, CD31, KDR, or p95.

In certain embodiments, the method comprises use of a VeraTag® assay. In certain embodiments, likeliness to respond is measured with respect to overall survival rate, time to progression and/or using the RECIST or other response criteria.

In certain embodiments, the method comprises detecting in a biological sample from the subject's cancer the amount of total HER1 and/or activated HER1, wherein if the amount of total HER1 and/or activated HER1 is high, then the patient is likely to respond to the HER1-acting agent and/or the patient has a long time course. In some embodiments, activated HER1 is measured.

In another aspect, the invention is drawn to a method for determining whether a subject with a HER1 positive cancer is likely to respond to treatment with a HER1-acting agent and/or have a long disease time course. In another aspect, the invention is drawn to a method for predicting a time course of disease in a subject with a HER1 positive cancer. In another aspect, the invention is drawn to a method for predicting the probability of a significant event in a subject with a HER1 positive cancer.

For example, the invention may comprise methods for predicting whether a subject with a cancer and being treated with a HER1-acting agent is likely to have a significant event comprising the steps of: (a) detecting in a biological sample from the subject's cancer the amount of total HER1 and/or activated HER1; and (b) correlating the amount of total HER1 and/or activated HER1 to the likelihood that the subject will have a significant event.

In one embodiment, the significant event is a reduced time between diagnosis with the cancer and at least one of progression of the cancer from one stage to a more advanced stage, progression to metastatic disease, relapse, surgery or death. Also in certain embodiments, the method may further comprise predicting a time course during which the significant event can occur.

In certain embodiments, if the amount of the activated HER1 is equal to or below a first threshold level, the significant event is that the subject is less likely to respond to the HER1-acting agent. Additionally, if the amount of the activated HER1 is equal to or above the first threshold level, the subject's prognosis is to be likely to respond to the HER1-acting agent.

In certain embodiments, a time course is measured by determining the time between significant events in the course of a patient's disease, wherein the measurement is predictive of whether a patient has a long time course. In one embodiment, the significant event is the progression from primary diagnosis to death. In another embodiment, the significant event is the progression from primary diagnosis to metastatic disease. In yet another embodiment, the significant event is the progression from primary diagnosis to relapse. In another embodiment, the significant event is the progression from metastatic disease to death. In another embodiment, the significant event is the progression from metastatic disease to relapse. In another embodiment, the significant event is the progression from relapse to death. In certain embodiments, the time course is measured with respect to overall survival rate, time to progression and/or using the RECIST or other response criteria.

In certain embodiments, the method comprises measuring in a biological sample from the subject's cancer an amount of activated HER1, wherein if the amount of activated HER1 is high, then the patient is likely to respond to the HER1-acting agent and/or the patient has a long time course. In certain embodiments, the biological sample comprises FFPE tissue. In certain embodiments, the subject's cancer is SCCHN. In certain embodiments, the SCCHN is metastatic or recurrent. In other embodiments, other cancers that may be sensitive to HER1-acting agents may be monitored. As noted herein, the HER1-acting agent may be any one of the HER1-acting agents known. In certain embodiments, the HER1-acting agent is cetuximab.

In certain embodiments, an amount of total HER1 is measured. In certain embodiments, an amount of activated HER1 is measured. In certain embodiments, the amount of activated HER1 is measured using an assay capable of measuring and/or quantifying an amount of protein-protein interactions in a sample. In certain embodiment, the assay is the VeraTag® assay. In certain embodiments, likeliness to respond is measured with respect to overall survival rate, time to progression, and/or using the RECIST criteria.

In certain embodiments, any method known to one of skill in the art to be useful for determining an amount of total HER1 expression and/or activated HER1 may be used. For example, any quantitative assay that determines the amount of HER1 expression can be used to determine how much signal is generated by a cell or cancer as representative of HER1 expression. In some embodiments, the signal from such an assay may be compared to the signal generated in the VeraTag® assay to determine a correspondence between the two assays. Such methods may include, but not necessarily be limited to, FRET, BRET, Biomolecular Fluoresence Complementation and Proximity Ligation Assay.

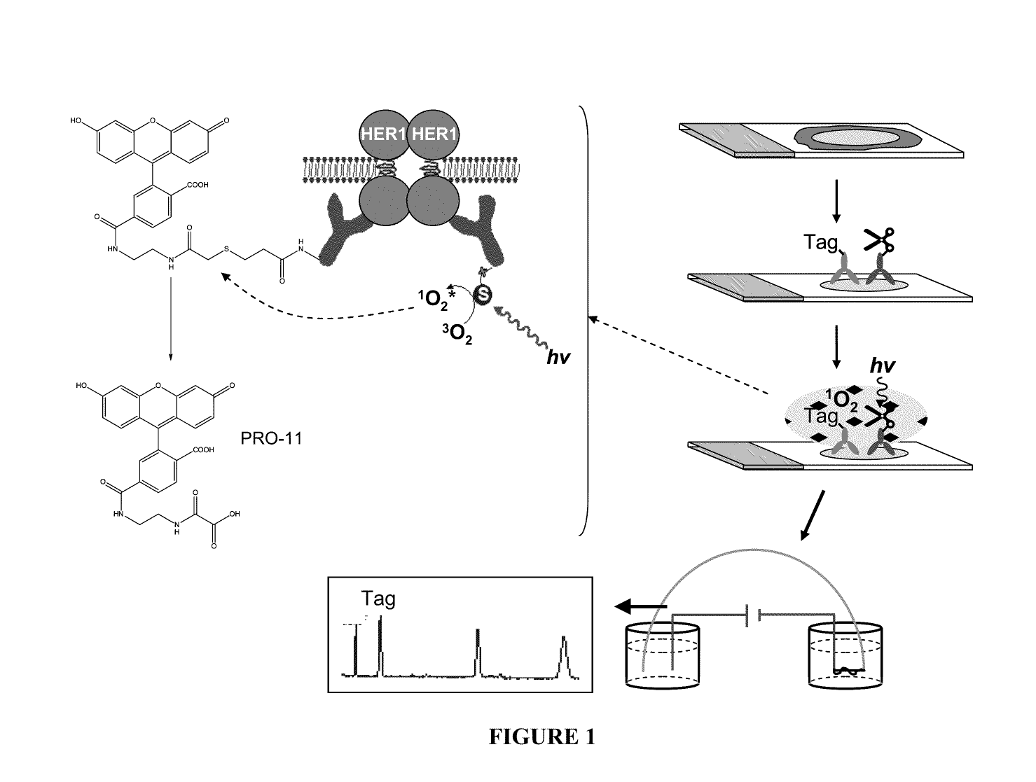

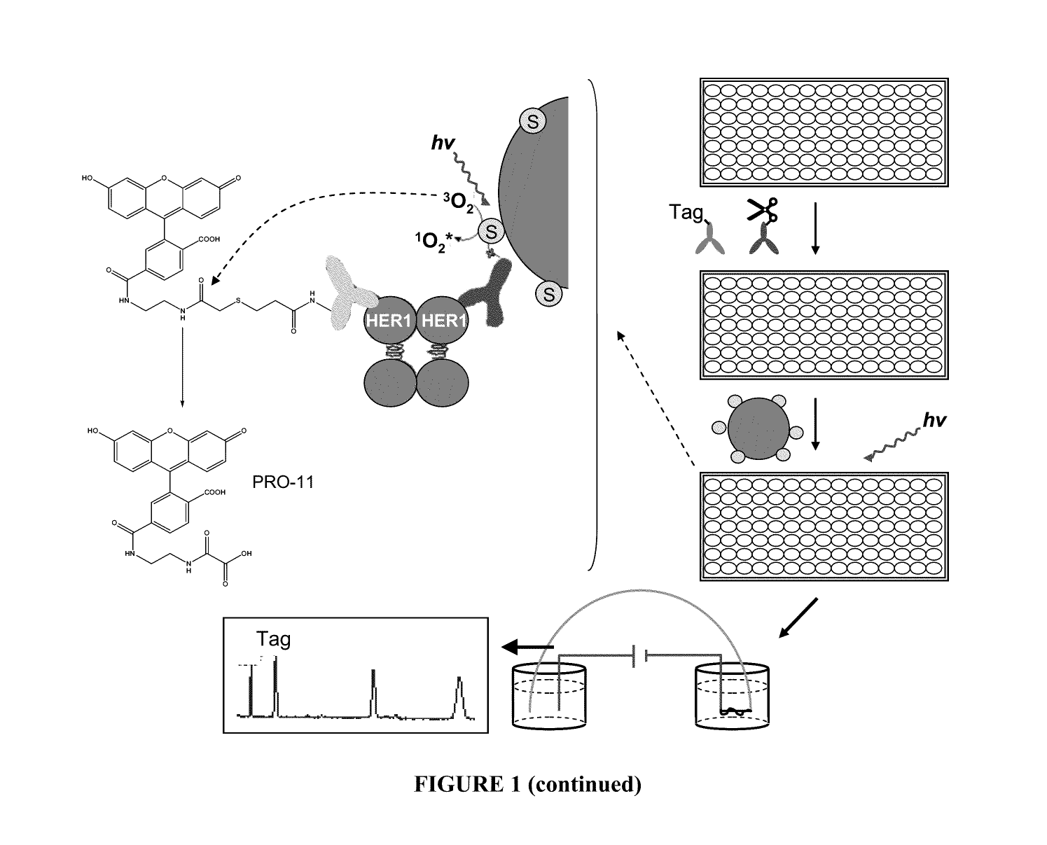

In certain embodiments, the amounts are determined by contacting a biological sample from a subject with cancer with a binding compound having a molecular tag attached thereto by a cleavable linkage and a cleaving probe having a cleavage inducing-moiety and detecting whether and what molecular tag is released. FIG. 1 provides a schematic diagram of such a VeraTag® assay. Tissue sections are fixed and then allowed to bind to a first antibody having a cleavage-inducing agent and a second antibody linked to a detectable moiety (e.g., VeraTag® reporter molecule). Here, a first antibody is conjugated to biotin, and a second antibody is conjugated to a VeraTag® reporter molecule. Streptavidin-functionalized sensitizer dye may then be bound to the biotin-conjugated antibody. Photo-induction of the cleavage-inducing agent may be performed to cause the release of singlet oxygen, which induces cleavage of the VeraTag® reporter molecule into the solution. The solution is then collected and analyzed by capillary electrophoresis. In some embodiments, cleavage of the VeraTag® reporter molecule may be caused by DTT instead of photosensitization induced cleavage.

In certain embodiments, the binding compound and the cleaving probe each specifically binds all HER1 or only activated HER1. For example, in certain embodiments, the binding compound specifically binds HER1 in a HER1 homodimer complex, HER1 in a HER1/HER2 heterodimer complex, or phosphorylated HER1. In certain embodiments, the cleaving probe and the binding probe do not both bind the same epitope. In certain embodiments, if the binding compound is within an effective proximity of the cleavage-inducing moiety of the cleaving probe, the cleavage-inducing moiety cleaves the cleavable linker so that the molecular tag is released. In certain embodiments, the molecular tag released if HER1 homodimers or HER1-HER2 heterodimers are present is distinguishable from the molecular tag released if HER1 monomers are present. Examples of detection of Her-2 by an assay for detection of total Her-2 and/or Her-2 homodimers is provided in commonly owned U.S. Patent Application Publication No. 2009/0191559 incorporated by reference in its entirety herein. A similar strategy can be used to measure other biomarkers such as HER1, HER3, cMET, p-95 and the like. See, e.g., U.S. Pat. No. 7,648,828 and U.S. Patent Application Nos. 2010/0143927; 2010/0233732; and 2010/0210034.

In certain embodiments, activating the cleavage-inducing moiety cleaves the cleavable linker. In certain embodiments, the binding compound specifically binds a HER1 epitope. In certain embodiments, the binding compound comprises an antibody or antigen-binding fragment. In certain embodiments, the binding compound specifically binds a HER1 ligand binding site. In certain embodiments, the binding compound comprises a HER1 ligand. In certain embodiments, the binding compound and the cleaving probe bind the same HER1 epitope.

In certain embodiments, one of more VeraTag® assay formats may be used to measure HER biomarkers levels. For example, when measuring total HER1 levels, a primary antibody that binds to the extracellular domain of HER1 may used. A secondary antibody with a VeraTag® reporter molecule attached thereto may then be used to specifically bind to the primary antibody. In certain embodiments of this assay format, the VeraTag® reporter molecule may be cleaved with a reducing agents such as, e.g., DTT.

When measuring amounts of total c-MET, HER2, HER3, and HER1 homodimers, two primary antibodies may be used, both primary antibodies specific for the target protein. One primary antibody may have a methylene blue dye attached, and bind to the intracellular domain of c-MET, HER2, or HER3. The second primary antibody is conjugated to a VeraTag® reporter molecule and may also bind the intracellular domain of c-MET, HER2, or HER3. Upon photosensitization, the methylene blue dye releases singlet oxygen which causes cleavage of the VeraTag® reporter molecule when the molecules are in close proximity. The two primary antibodies may be identical or may bind to two different epitopes of the HER molecule. When measuring HER1 phosphorylation at position 1173, the methylene blue conjugated antibody may bind to HER1 phosphorylated at position 1173. When measuring HER1pan phosphorylation, the VeraTag® reporter molecule-conjugated antibody may bind to a phosphorylated epitope.

When measuring HER1-HER2 heterodimers, two different primary antibodies that bind the extracellular domain of HER1 or HER2, respectively, as well as two secondary antibodies that specifically bind to one or the other primary antibodies, may be used. For example, one secondary antibody may be conjugated to the VeraTag® reporter molecule and bind the HER2 primary antibody. In this embodiment, the second secondary antibody may then be conjugated to methylene blue and bind to the HER1 primary antibody. Upon photosensitization, if the secondary antibodies are located in close proximity to one another, the VeraTag® reporter molecule may be cleaved. Subsequently, the cleaved reporter molecules may then separated, for example, by electrophoresis.

The invention relates to HER1-acting agents. A HER1-acting agent can be any such agent known to one of skill in the art. In certain embodiments the HER1-acting agent is selected from the group consisting of cetuximab (ERBITUX), gefitinib (IRESSA), erlotinib (TARCEVA), lapatinib (TYKERB), and panitumumab (VECTIBIX). In a preferred embodiment, the Her-2-acting agent is cetuximab (ERBITUX). Also, other HER1-acting agents may be evaluated using the methods described herein.

Samples containing HER1 or activated HER1 suitable for use as biomarkers may come from a wide variety of sources, including cell cultures, animal tissues, patient biopsies or the like. Preferably, samples are human patient samples. Samples are prepared for assays of the invention using conventional techniques, which may depend on the source from which a sample is taken. For biopsies and medical specimens, guidance is provided in the following references: Bancroft J D & Stevens A, eds. 1977, Theory and Practice of Histological Techniques, Churchill Livingstone, Edinburgh; Pearse, 1980, Histochemistry. Theory and applied. 4^{th} ed., Churchill Livingstone, Edinburgh.

In the area of cancerous disease status, examples of patient tissue samples that may be used include, but are not limited to, breast, prostate, ovary, colon, lung, endometrium, stomach, salivary gland, larynx, pharynx, tongue, or pancreas. The tissue sample can be obtained by a variety of procedures including surgical excision, aspiration or biopsy. The tissue may be fresh or frozen. In one embodiment, assays of the invention are carried out on tissue samples that have been fixed and embedded in paraffin and a step of deparaffination is be carried out. A tissue sample may be fixed (i.e., preserved) by conventional methodology. See, e.g., Lee G. Luna, H T (ASCP) Ed., 1960, Manual of Histological Staining Method of the Armed Forces Institute of Pathology 3^{rd} edition, The Blakston Division McGraw-Hill Book Company, New York; Ulreka V. Mikel, Ed., 1994, The Armed Forces Institute of Pathology Advanced Laboratory Methods in Histology and Pathology, Armed Forces Institute of Pathology, American Registry of Pathology, Washington, D.C. One of skill in the art will appreciate that the choice of a fixative is determined by the purpose for which the tissue is to be histologically stained or otherwise analyzed. One of skill in the art will also appreciate that the length of fixation depends upon the size of the tissue sample and the fixative used.

Generally, a tissue sample is first fixed and is then dehydrated through an ascending series of alcohols, infiltrated and embedded with paraffin or other sectioning media so that the tissue sample may be sectioned. Alternatively, one may section the tissue and fix the sections obtained. By way of example, the tissue sample may be embedded and processed in paraffin by conventional methodology according to conventional techniques described by the references provided above. Examples of paraffin that may be used include, but are not limited to, Paraplast, Broloid, and Tissuemay. Once the tissue sample is embedded, the sample may be sectioned by a microtome according to conventional techniques. Sections may have a thickness in a range of about three microns to about twelve microns, and preferably, a thickness in a range of about 5 microns to about 10 microns. In one aspect, a section may have an area of about 10 mm² to about 1 cm². Once cut, the sections may be attached to slides by several standard methods. Examples of slide adhesives include, but are not limited to, silane, gelatin and poly-L-lysine. Paraffin embedded sections may be attached to positively charged slides and/or slides coated with poly-L-lysine.

If paraffin has been used as the embedding material, the tissue sections are generally deparaffinized and rehydrated to water prior to detection of biomarkers. Tissue sections may be deparaffinized by several conventional standard methodologies. For example, xylenes and a gradually descending series of alcohols may be used according to conventional techniques described by the references provided above. Alternatively, commercially available deparaffinizing non-organic agents such as Hemo-De® (CMS, Houston, Tex.) may be used.

Mammalian tissue culture cells, or fresh or frozen tissues may be prepared by conventional cell lysis techniques (e.g., 0.14 M NaCl, 1.5 mM MgCl₂, 10 mM Tris-Cl (pH 8.6), 0.5% Nonidet P-40, and protease and/or phosphatase inhibitors as required). For fresh mammalian tissues, sample preparation may also include a tissue disaggregation step, such as crushing, mincing, grinding or sonication.

Many advantages are provided by measuring activated HER1 populations using releasable molecular tags, including (1) separation of released molecular tags from an assay mixture provides greatly reduced background and a significant gain in sensitivity; and (2) the use of molecular tags that are specially designed for ease of separation and detection provides a convenient multiplexing capability so that multiple receptor complex components may be readily measured simultaneously in the same assay. Assays employing such tags can have a variety of forms and are disclosed in the following references: U.S. Pat. Nos. 7,105,308 and 6,627,400; published U.S. Patent Application Nos. 2002/0013126, 2003/0170915, 2002/0146726, 2009/0191559, 2010/0143927, 2010/0233732, and 2010/0210034; and International Patent Publication No. WO 2004/011900, each of which are incorporated herein by reference in their entireties. For example, a wide variety of separation techniques may be employed that can distinguish molecules based on one or more physical, chemical or optical differences among molecules being separated including electrophoretic mobility, molecular weight, shape, solubility, pKa, hydrophobicity, charge, charge/mass ratio or polarity. In one aspect, molecular tags in a plurality or set differ in electrophoretic mobility and optical detection characteristics and are separated by electrophoresis. In another aspect, molecular tags in a plurality or set may differ in molecular weight, shape, solubility, pKa, hydrophobicity, charge, polarity and are separated by normal phase or reverse phase HPLC, ion exchange HPLC, capillary electrochromatography, mass spectroscopy or gas phase chromatography.

Sets of molecular tags are provided that can be separated into distinct bands or peaks by a separation technique after they are released from binding compounds. Identification and quantification of such peaks provides a measure or profile of the presence and/or amounts of proteins, protein complexes and post-translationally modified proteins. Molecular tags within a set may be chemically diverse; however, for convenience, sets of molecular tags are usually chemically related. For example, they may all be peptides or they may consist of different combinations of the same basic building blocks or monomers or they may be synthesized using the same basic scaffold with different substituent groups for imparting different separation characteristics. The number of molecular tags in a plurality may vary depending on several factors including the mode of separation employed, the labels used on the molecular tags for detection, the sensitivity of the binding moieties and the efficiency with which the cleavable linkages are cleaved.

Measurements made directly on tissue samples may be normalized by including measurements on cellular or tissue targets that are representative of the total cell number in the sample and/or the numbers of particular subtypes of cells in the sample. The additional measurement may be preferred, or even necessary, because of the cellular and tissue heterogeneity in patient samples, particularly tumor samples, which may comprise substantial fractions of normal cells.

As mentioned above, mixtures containing pluralities of different binding compounds may be provided, wherein each different binding compound has one or more molecular tags attached through cleavable linkages. The nature of the binding compound, cleavable linkage and molecular tag may vary widely. A binding compound may comprise an antibody binding composition, an antibody, a peptide, a peptide or non-peptide ligand for a cell surface receptor, a protein, an oligonucleotide, an oligonucleotide analog, such as a peptide nucleic acid, a lectin or any other molecular entity that is capable of specifically binding to a target protein or molecule or stable complex formation with an analyte of interest, such as an activated HER1. In one aspect, a binding compound can be represented by the following formula:

B-(L-E)ₖ

wherein B is binding moiety; L is a cleavable linkage, and E is a molecular tag. In homogeneous assays, cleavable linkage, L, may be an oxidation-labile linkage, and more preferably, it is a linkage that may be cleaved by singlet oxygen. Alternatively, it may be a linkage that is sensitive to cleavage by reduction, for example by DTT. The moiety “-(L-E)ₖ” indicates that a single binding compound may have multiple molecular tags attached via cleavable linkages. In one aspect, k is an integer greater than or equal to one, but in other embodiments, k may be greater than several hundred, e.g., 100 to 500 or k is greater than several hundred to as many as several thousand, e.g., 500 to 5000. Usually each of the plurality of different types of binding compounds has a different molecular tag, E. Cleavable linkages, e.g., oxidation-labile linkages, and molecular tags, E, are attached to B by way of conventional chemistries.

Preferably, B is an antibody binding composition that specifically binds to a target, such as an antigenic determinant on HER1. Antibodies specific for HER1 epitopes are provided in the examples set forth herein. Antibody compositions are readily formed from a wide variety of commercially available antibodies, either monoclonal or polyclonal. In particular, antibodies specific for epidermal growth factor receptors are disclosed in U.S. Pat. Nos. 5,677,171; 5,772,997; 5,968,511; 5,480,968; 5,811,098, each of which are incorporated by reference in its entirety. U.S. Pat. No. 5,599,681, hereby incorporated by reference in its entirety, discloses antibodies specific for phosphorylation sites of proteins. Commercial vendors, such as e.g., Cell Signaling Technology (Beverly, Mass.), Biosource International (Camarillo, Calif.) and Upstate (Charlottesville, Va.), also provide monoclonal and polyclonal antibodies.

Cleavable linkage, L, can be virtually any chemical linking group that may be cleaved under conditions that do not degrade the structure or affect detection characteristics of the released molecular tag, E. In certain embodiments, a cleaving probe used in a homogeneous assay format, has a cleavable linkage, L, cleaved by a cleavage agent generated by the cleaving probe that acts over a short distance so that only cleavable linkages in the immediate proximity of the cleaving probe are cleaved. Typically, such an agent must be activated by making a physical or chemical change to the reaction mixture so that the agent produces a short lived active species that diffuses to a cleavable linkage to effect cleavage. In a homogeneous format, the cleavage agent is preferably attached to a binding moiety, such as an antibody, that targets prior to activation the cleavage agent to a particular site in the proximity of a binding compound with releasable molecular tags. In such embodiments, a cleavage agent is referred to herein as a “cleavage-inducing moiety.”

In a non-homogeneous format, because specifically bound binding compounds are separated from unbound binding compounds, a wider selection of cleavable linkages and cleavage agents are available for use. Cleavable linkages may not only include linkages that are labile to reaction with a locally acting reactive species, such as hydrogen peroxide, singlet oxygen or the like, but also linkages that are labile to agents that operate throughout a reaction mixture, such as base-labile linkages, photocleavable linkages, linkages cleavable by reduction, linkages cleaved by oxidation, acid-labile linkages and peptide linkages cleavable by specific proteases. References describing many such linkages include Greene and Wuts, 1991, Protective Groups in Organic Synthesis, Second Edition, John Wiley & Sons, New York; Hermanson, 1996, Bioconjugate Techniques, Academic Press, New York; and U.S. Pat. No. 5,565,324.

In one aspect, commercially available cleavable reagent systems may be employed with the invention. For example, a disulfide linkage may be introduced between an antibody binding composition and a molecular tag using a heterofunctional agent such as N-succinimidyl 3-(2-pyridyldithio)propionate (SPDP), succinimidyloxycarbonyl-\acute{α}-methyl-\acute{α}-(2-pyridyldithio) toluene (SMPT) or the like, available from vendors such as Pierce Chemical Company (Rockford, Ill.). Disulfide bonds introduced by such linkages can be broken by treatment with a reducing agent, such as dithiothreitol (DTT), dithioerythritol (DTE), 2-mercaptoethanol or sodium borohydride. Typical concentrations of reducing agents to effect cleavage of disulfide bonds are in the range of about 1 mM to 100 mM. An oxidatively labile linkage may be introduced between an antibody binding composition and a molecular tag using the homobifunctional NHS ester cross-linking reagent, disuccinimidyl tartarate (DST) (available from Pierce) that contains central cis-diols that are susceptible to cleavage with sodium periodate (e.g., 15 mM periodate at physiological pH for 4 hours). Linkages that contain esterified spacer components may be cleaved with strong nucleophilic agents, such as hydroxylamine, e.g., 0.1 N hydroxylamine, pH 8.5, for 3-6 hours at 37° C. Such spacers can be introduced by a homobifunctional cross-linking agent such as ethylene glycol bis(succinimidylsuccinate) (EGS) available from Pierce (Rockford, Ill.). A base labile linkage can be introduced with a sulfone group. Homobifunctional cross-linking agents that can be used to introduce sulfone groups in a cleavable linkage include bis[2-(succinimidyloxycarbonyloxy)ethyl]sulfone (BSOCOES), and 4,4-difluoro-3,3-dinitrophenyl-sulfone (DFDNPS). Exemplary basic conditions for cleavage include 0.1 M sodium phosphate, adjusted to pH 11.6 by addition of Tris base, containing 6 M urea, 0.1% SDS, and 2 mM DTT, with incubation at 37° C. for 2 hours. Photocleavable linkages also include those disclosed in U.S. Pat. No. 5,986,076.

When L is oxidation labile, L may be a thioether or its selenium analog; or an olefin, which contains carbon-carbon double bonds, wherein cleavage of a double bond to an oxo group, releases the molecular tag, E. Illustrative oxidation labile linkages are disclosed in U.S. Pat. Nos. 6,627,400 and 5,622,929 and in published U.S. Patent Application Nos. 2002/0013126 and 2003/0170915; each of which is hereby incorporated herein by reference in its entirety.

Molecular tag, E, in the present invention may comprise an electrophoric tag as described in the following references when separation of pluralities of molecular tags are carried out by gas chromatography or mass spectrometry: See, e.g., Zhang et al., 2002, Bioconjugate Chem. 13:1002-1012; Giese, 1983, Anal. Chem. 2:165-168; and U.S. Pat. Nos. 4,650,750; 5,360,819; 5,516,931; and 5,602,273, each of which is hereby incorporated by reference in its entirety.

Molecular tag, E, is preferably a water-soluble organic compound that is stable with respect to the active species, especially singlet oxygen, and that includes a detection or reporter group. Otherwise, E may vary widely in size and structure. In one aspect, E has a molecular weight in the range of about 50 to about 2500 daltons, more preferably, from about 50 to about 1500 daltons. E may comprise a detection group for generating an electrochemical, fluorescent or chromogenic signal. In embodiments employing detection by mass, E may not have a separate moiety for detection purposes. Preferably, the detection group generates a fluorescent signal.

Molecular tags within a plurality are selected so that each has a unique separation characteristic and/or a unique optical property with respect to the other members of the same plurality. In one aspect, the chromatographic or electrophoretic separation characteristic is retention time under a set of standard separation conditions conventional in the art, e.g., voltage, column pressure, column type, mobile phase or electrophoretic separation medium. In another aspect, the optical property is a fluorescence property, such as emission spectrum, fluorescence lifetime or fluorescence intensity at a given wavelength or band of wavelengths. Preferably, the fluorescence property is fluorescence intensity. For example, each molecular tag of a plurality may have the same fluorescent emission properties, but each will differ from one another by virtue of a unique retention time. On the other hand, one or two or more of the molecular tags of a plurality may have identical migration or retention times, but they will have unique fluorescent properties, e.g., spectrally resolvable emission spectra, so that all the members of the plurality are distinguishable by the combination of molecular separation and fluorescence measurement.

Preferably, released molecular tags are detected by electrophoretic separation and the fluorescence of a detection group. In such embodiments, molecular tags having substantially identical fluorescence properties have different electrophoretic mobilities so that distinct peaks in an electropherogram are formed under separation conditions. Preferably, pluralities of molecular tags of the invention are separated by conventional capillary electrophoresis apparatus, either in the presence or absence of a conventional sieving matrix. During or after electrophoretic separation, the molecular tags are detected or identified by recording fluorescence signals and migration times (or migration distances) of the separated compounds or by constructing a chart of relative fluorescent and order of migration of the molecular tags (e.g., as an electropherogram). Preferably, the presence, absence and/or amounts of molecular tags are measured by using one or more standards as disclosed by published U.S. Patent Application No. 2003/0170734A1, which is hereby incorporated by reference in its entirety.

Pluralities of molecular tags may also be designed for separation by chromatography based on one or more physical characteristics that include molecular weight, shape, solubility, pKa, hydrophobicity, charge, polarity or the like, e.g., as disclosed in published U.S. Pat. No. 7,255,999, which hereby is incorporated by reference in its entirety. A chromatographic separation technique is selected based on parameters such as column type, solid phase, mobile phase and the like, followed by selection of a plurality of molecular tags that may be separated to form distinct peaks or bands in a single operation. Several factors determine which HPLC technique is selected for use in the invention, including the number of molecular tags to be detected (i.e., the size of the plurality), the estimated quantities of each molecular tag that will be generated in the assays, the availability and ease of synthesizing molecular tags that are candidates for a set to be used in multiplexed assays, the detection modality employed and the availability, robustness, cost and ease of operation of HPLC instrumentation, columns and solvents. Generally, columns and techniques are favored that are suitable for analyzing limited amounts of sample and that provide the highest resolution separations. Guidance for making such selections can be found in the literature, such as, for example, Snyder et al., 1988, Practical HPLC Method Development, John Wiley & Sons, New York; Millner, 1999, High Resolution Chromatography: A Practical Approach, Oxford University Press, New York; Chi-San Wu, 1999, Column Handbook for Size Exclusion Chromatography, Academic Press, San Diego; and Oliver, 1989, HPLC of Macromolecules: A Practical Approach, Oxford University Press, Oxford, England.

In one aspect, molecular tag, E, is (M, D), where M is a mobility-modifying moiety and D is a detection moiety. The notation “(M, D)” is used to indicate that the ordering of the M and D moieties may be such that either moiety can be adjacent to the cleavable linkage, L. That is, “B-L-(M, D)” designates binding compound of either of two forms: “B-L-M-D” or “B-L-D-M.”

Detection moiety, D, may be a fluorescent label or dye, a chromogenic label or dye or an electrochemical label. Preferably, D is a fluorescent dye. Exemplary fluorescent dyes for use with the invention include water-soluble rhodamine dyes, fluoresceins, 4,7-dichlorofluoresceins, benzoxanthene dyes and energy transfer dyes, as disclosed in the following references: Handbook of Molecular Probes and Research Reagents, 8^{th} ed. (2002), Molecular Probes, Eugene, Oreg.; U.S. Pat. Nos. 6,191,278, 6,372,907, 6,096,723, 5,945,526, 4,997,928, and 4,318,846; and Lee et al., 1997, Nucleic Acids Research 25:2816-2822. Preferably, D is a fluorescein or a fluorescein derivative.

Once each of the binding compounds is separately derivatized by a different molecular tag, it is pooled with other binding compounds to form a plurality of binding compounds. Usually, each different kind of binding compound is present in a composition in the same proportion; however, proportions may be varied as a design choice so that one or a subset of particular binding compounds are present in greater or lower proportion depending on the desirability or requirements for a particular embodiment or assay. Factors that may affect such design choices include, but are not limited to, antibody affinity and avidity for a particular target, relative prevalence of a target, fluorescent characteristics of a detection moiety of a molecular tag and the like.

A cleavage-inducing moiety, or cleaving agent, is a group that produces an active species that is capable of cleaving a cleavable linkage, preferably by oxidation. Preferably, the active species is a chemical species that exhibits short-lived activity so that its cleavage-inducing effects are only in the proximity of the site of its generation. Either the active species is inherently short lived, so that it will not create significant background beyond the proximity of its creation, or a scavenger is employed that efficiently scavenges the active species, so that it is not available to react with cleavable linkages beyond a short distance from the site of its generation. Illustrative active species include singlet oxygen, hydrogen peroxide, NADH, and hydroxyl radicals, phenoxy radical, superoxide and the like. Illustrative quenchers for active species that cause oxidation include polyenes, carotenoids, vitamin E, vitamin C, amino acid-pyrrole N-conjugates of tyrosine, histidine and glutathione. See, e.g., Beutner et al., 2000, Meth. Enzymol. 319:226-241.

One consideration in designing assays employing a cleavage-inducing moiety and a cleavable linkage is that they not be so far removed from one another when bound to a receptor complex that the active species generated by the cleavage-inducing moiety cannot efficiently cleave the cleavable linkage. In one aspect, cleavable linkages preferably are within about 1000 nm and preferably within about 20-200 nm, of a bound cleavage-inducing moiety. More preferably, for photosensitizer cleavage-inducing moieties generating singlet oxygen, cleavable linkages are within about 20-100 nm of a photosensitizer in a receptor complex. The range within which a cleavage-inducing moiety can effectively cleave a cleavable linkage (that is, cleave enough molecular tag to generate a detectable signal) is referred to herein as its “effective proximity.” One of ordinary skill in the art will recognize that the effective proximity of a particular sensitizer may depend on the details of a particular assay design and may be determined or modified by routine experimentation.

A sensitizer is a compound that can be induced to generate a reactive intermediate, or species, usually singlet oxygen. Preferably, a sensitizer used in accordance with the invention is a photosensitizer. Other sensitizers included within the scope of the invention are compounds that on excitation by heat, light, ionizing radiation or chemical activation will release a molecule of singlet oxygen. The best known members of this class of compounds include the endoperoxides such as 1,4-biscarboxyethyl-1,4-naphthalene endoperoxide, 9,10-diphenylanthracene-9,10-endoperoxide and 5,6,11,12-tetraphenyl naphthalene 5,12-endoperoxide. Heating or direct absorption of light by these compounds releases singlet oxygen. Further sensitizers are disclosed by Di Mascio et al., 1994, FEBS Lett. 355:287; and Kanofsky, 1983, J. Biol. Chem. 258:5991-5993; Pierlot et al., 2000, Meth. Enzymol. 319:3-20.

Photosensitizers may be attached directly or indirectly, via covalent or non-covalent linkages, to the binding agent of a class-specific reagent. Guidance for constructing such compositions, particularly for antibodies as binding agents are available in the literature, e.g., in the fields of photodynamic therapy, immunodiagnostics, and the like. Exemplary guidance may be found in Ullman et al., 1994, Proc. Natl. Acad. Sci. USA 91, 5426-5430; Strong et al., 1994, Ann. New York Acad. Sci. 745: 297-320; Yarmush et al., 1993, Crit. Rev. Therapeutic Drug Carrier Syst. 10: 197-252; and U.S. Pat. Nos. 5,340,716, 5,516,636, 5,709,994, and 6,251,581.

A large variety of light sources are available to photo-activate photosensitizers to generate singlet oxygen. Both polychromatic and monochromatic sources may be used as long as the source is sufficiently intense to produce enough singlet oxygen in a practical time duration. The length of the irradiation depends on the nature of the photosensitizer, the nature of the cleavable linkage, the power of the source of irradiation and its distance from the sample. In general, the period for irradiation may be more than about a microsecond to as long as about 10 minutes, usually in the range of about one millisecond to about 60 seconds. The intensity and length of irradiation should be sufficient to excite at least about 0.1% of the photosensitizer molecules, usually at least about 30% of the photosensitizer molecules and preferably, substantially all of the photosensitizer molecules. Exemplary light sources include lasers such as, e.g., helium-neon lasers, argon lasers, YAG lasers, He/Cd lasers and ruby lasers; photodiodes; mercury, sodium and xenon vapor lamps; incandescent lamps such as, e.g., tungsten and tungsten/halogen and flashlamps. An exemplary photoactivation device suitable for use in the methods of the invention is disclosed International Patent Publication No. WO 03/051669. In such embodiments, the photoactivation device is an array of light emitting diodes (LEDs) mounted in housing that permits the simultaneous illumination of all the wells in a 96-well plate.

Examples of photosensitizers that may be utilized in the present invention are those that have the above properties and those disclosed by U.S. Pat. Nos. 5,536,834, 5,763,602, 5,565,552, 5,709,994, 5,340,716, 5,516,636, 6,251,581, and 6,001,673; published European Patent Application No. 0484027; Martin et al., 1990, Methods Enzymol. 186:635-645; and Yarmush et al., 1993, Crit. Rev. Therapeutic Drug Carrier Syst. 10:197-252. As with sensitizers, in certain embodiments, a photosensitizer may be associated with a solid phase support by being covalently or non-covalently attached to the surface of the support or incorporated into the body of the support. In general, the photosensitizer is associated with the support in an amount necessary to achieve the necessary amount of singlet oxygen. Generally, the amount of photosensitizer is determined empirically according to routine methods.

In one embodiment, a photosensitizer is incorporated into a latex particle to form photosensitizer beads, e.g., as disclosed by U.S. Pat. Nos. 5,709,994 and 6,346,384; and International Patent Publication No. WO 01/84157. Alternatively, photosensitizer beads may be prepared by covalently attaching a photosensitizer, such as rose bengal, to 0.5 micron latex beads by means of chloromethyl groups on the latex to provide an ester linking group, as described in J. Amer. Chem. Soc., 97:3741 (1975). Methods employing latex particle, photosensitizer beads may be carried out, for example, in a conventional 96-well or 384-well microtiter plate, or the like, having a filter membrane that forms one wall, e.g., the bottom, of the wells that allows reagents to be removed by the application of a vacuum. This allows the convenient exchange of buffers, if the buffer required for specific binding of binding compounds is different than the buffer required for either singlet oxygen generation or separation. For example, in the case of antibody-based binding compounds, a high salt buffer is required. If electrophoretic separation of the released tags is employed, then better performance is achieved by exchanging the buffer for one that has a lower salt concentration suitable for electrophoresis.

As an example, a cleaving probe may comprise a primary haptenated antibody and a secondary anti-hapten binding protein derivatized with multiple photosensitizer molecules. A preferred primary haptenated antibody is a biotinylated antibody and preferred secondary anti-hapten binding proteins may be either an anti-biotin antibody or streptavidin. Other combinations of such primary and secondary reagents are well known in the art. Exemplary combinations of such reagents are taught by Haugland, 2002, Handbook of Fluorescent Probes and Research Reagents, Ninth Edition, Molecular Probes, Eugene, Oreg. An exemplary combination of such reagents is described below. There binding compounds having releasable tags (“mT₁” and “mT₂”), and primary antibody derivatized with biotin are specifically bound to different epitopes of receptor dimer in membrane. Biotin-specific binding protein, e.g., streptavidin, is attached to biotin bringing multiple photosensitizers into effective proximity of binding compounds. Biotin-specific binding protein may also be an anti-biotin antibody and photosensitizers may be attached via free amine group on the protein by conventional coupling chemistries, e.g., Hermanson (supra). An exemplary photosensitizer for such use is an NHS ester of methylene blue prepared as disclosed in published European Patent Application 0510688.

The following general discussion of methods and specific conditions and materials are by way of illustration and not limitation. One of skill in the art will understand how the methods described herein can be adapted to other applications, particularly with using different samples, cell types, and target complexes.

In conducting the methods of the invention, a combination of the assay components is made, including the sample being tested, the binding compounds and optionally the cleaving probe. Generally, assay components may be combined in any order. In certain applications, however, the order of addition may be relevant. For example, one may wish to quantitatively monitor competitive binding. Or one may wish to monitor the stability of an assembled complex. In such applications, reactions may be assembled in stages.

The amounts of each reagent can generally be determined empirically. The amount of sample used in an assay will be determined by the predicted number of target complexes present and the means of separation and detection used to monitor the signal of the assay. In general, the amounts of the binding compounds and the cleaving probe can be provided in molar excess relative to the expected amount of the target molecules in the sample, generally at a molar excess of at least about 1.5, more desirably about 10-fold excess, or more. In specific applications, the concentration used may be higher or lower, depending on the affinity of the binding agents and the expected number of target molecules present on a single cell. Where one is determining the effect of a chemical compound on formation of oligomeric cell surface complexes, the compound may be added to the cells prior to, simultaneously with, or after addition of the probes, depending on the effect being monitored.

The assay mixture can be combined and incubated under conditions that provide for binding of the probes to the cell surface molecules, usually in an aqueous medium, generally at a physiological pH (comparable to the pH at which the cells are cultures), maintained by a buffer at a concentration in the range of about 10 to 200 mM. Conventional buffers may be used, as well as other conventional additives as necessary, such as salts, growth medium, stabilizers, etc. Physiological and constant temperatures are normally employed. Incubation temperatures normally range from about 4° to 70° C., usually from about 15° to 45° C., more usually about 25° to 37° C.

After assembly of the assay mixture and incubation to allow the probes to bind to cell surface molecules, the mixture can be treated to activate the cleaving agent to cleave the tags from the binding compounds that are within the effective proximity of the cleaving agent, releasing the corresponding tag from the cell surface into solution. The nature of this treatment will depend on the mechanism of action of the cleaving agent. For example, where a photosensitizer is employed as the cleaving agent, activation of cleavage can comprise irradiation of the mixture at the wavelength of light appropriate to the particular sensitizer used.

Following cleavage, the sample can then be analyzed to determine the identity of tags that have been released. Where an assay employing a plurality of binding compounds is employed, separation of the released tags will generally precede their detection. The methods for both separation and detection are determined in the process of designing the tags for the assay. A preferred mode of separation employs electrophoresis, in which the various tags are separated based on known differences in their electrophoretic mobilities.

As mentioned above, in some embodiments, if the assay reaction conditions may interfere with the separation technique employed, it may be necessary to remove, or exchange, the assay reaction buffer prior to cleavage and separation of the molecular tags. For example, assay conditions may include salt concentrations (e.g., required for specific binding) that degrade separation performance when molecular tags are separated on the basis of electrophoretic mobility. Thus, such high salt buffers may be removed, e.g., prior to cleavage of molecular tags, and replaced with another buffer suitable for electrophoretic separation through filtration, aspiration, dilution or other means.

In certain embodiments, the subject may be administered a combination therapy that includes a HER1-acting agent such as cetuximab. The combination therapy can include the HER1-acting agent in combination with one or more of any chemotherapeutic agent known to one of skill in the art without limitation. Preferably, the chemotherapeutic agent has a different mechanism of action from the HER1-acting agent. For example, the chemotherapeutic agent can be an anti-metabolite (e.g., 5-fluorouricil (5-FU), methotrexate (MTX), fludarabine, etc.), an antimicrotubule agent (e.g., vincristine; vinblastine; taxanes such as paclitaxel and docetaxel; etc.), an alkylating agent (e.g., cyclophosphamide, melphalan, bischloroethylnitrosurea, etc.), platinum agents (e.g., cisplatin, carboplatin, oxaliplatin, JM-216, CI-973, etc.), anthracyclines (e.g., doxorubicin, daunorubicin, etc.), antibiotic agents (e.g., mitomycin-C, actinomycin D, etc.), topoisomerase inhibitors (e.g., etoposide, camptothecins, etc.) or other any other chemotherapeutic agents known to one skilled in the art.

Particular examples of chemotherapeutic agents that can be used in the various embodiments of the invention, including pharmaceutical compositions, dosage forms, and kits of the invention, include, without limitation, cytarabine, melphalan, topotecan, fludarabine, etoposide, idarubicin, daunorubicin, mitoxantrone, cisplatin paclitaxel, and cyclophosphamide.

Other chemotherapeutic agents that may be used include abarelix, aldesleukin, alemtuzumab, alitretinoin, allopurinol, altretamine, amifostine, anastrozole, arsenic trioxide, asparaginase, BCG live, bevaceizumab, bexarotene, bleomycin, bortezomib, busulfan, calusterone, camptothecin, capecitabine, carboplatin, carmustine, celecoxib, cetuximab, chlorambucil, cinacalcet, cisplatin, cladribine, cyclophosphamide, cytarabine, dacarbazine, dactinomycin, darbepoetin alfa, daunorubicin, denileukin diftitox, dexrazoxane, docetaxel, doxorubicin, dromostanolone, Elliott's B solution, epirubicin, epoetin alfa, estramustine, etoposide, exemestane, filgrastim, floxuridine, fludarabine, fluorouracil, fulvestrant, gemcitabine, gemtuzumab ozogamicin, gefitinib, goserelin, hydroxyurea, ibritumomab tiuxetan, idarubicin, ifosfamide, imatinib, interferon alfa-2a, interferon alfa-2b, irinotecan, letrozole, leucovorin, levamisole, lomustine, meclorethamine, megestrol, melphalan, mercaptopurine, mesna, methotrexate, methoxsalen, methylprednisolone, mitomycin C, mitotane, mitoxantrone, nandrolone, nofetumomab, oblimersen, oprelvekin, oxaliplatin, paclitaxel, pamidronate, pegademase, pegaspargase, pegfilgrastim, pemetrexed, pentostatin, pipobroman, plicamycin, polifeprosan, porfimer, procarbazine, quinacrine, rasburicase, rituximab, sargramostim, streptozocin, talc, tamoxifen, tarceva, temozolomide, teniposide, testolactone, thioguanine, thiotepa, topotecan, toremifene, tositumomab, trastuzumab, tretinoin, uracil mustard, valrubicin, vinblastine, vincristine, vinorelbine, and zoledronate.

In another aspect, the invention is drawn to a method for determining whether a subject with a HER1 positive cancer is unlikely to respond to treatment with at least one chemotherapeutic agent in addition to a HER1-acting agent and/or the patient is likely to have a short time course. In certain embodiments, the method comprises measuring in a biological sample from the subject's cancer an amount of HER1 and/or activated HER1, wherein if the level of HER1 and/or activated HER1 is high or very high, then the patient is unlikely to respond to at least one chemotherapeutic agent in addition to a HER1 acting agent.

In certain embodiments, an amount of activated HER1 is measured. In certain embodiments, an amount of HER1 homodimers, HER1/HER2 heterodimers, or phosphorylated HER1 is measured. In certain embodiments, more than one of these molecules are measured. In certain embodiments, the amount of activated HER1 is measured using an assay capable of measuring and/or quantifying an amount of protein-protein interactions in a sample. In a certain embodiment, the assay is the VeraTag® assay. In certain embodiments, likeliness to respond is measured with respect to overall survival rate, time to progression and/or using the RECIST criteria or other response criteria.

In another aspect, the invention is drawn to a method for determining whether a subject with a HER1 positive cancer is likely to respond to treatment with at least one chemotherapeutic agent in addition to a HER1-acting agent. In certain embodiments, the method comprises measuring in a biological sample from the subject's cancer an amount of activated HER1, wherein if the level of activated HER1 is high, then the patient is likely to respond to at least one chemotherapeutic agent in addition to the HER1-acting agent. In certain embodiments, the biological sample comprises FFP tissues. In certain embodiments, the subject's cancer is SCCHN. In certain embodiments, the cancer is metastatic or recurrent. In some embodiments, any cancer that may be sensitive to a HER1-acting agent may be monitored. The HER1-acting agent may be any HER1-acting agent. In certain embodiments, the HER1-acting agent is one of the agents described herein. Alternatively, other additional chemotherapeutic agents as known in the art and/or disclosed herein may be evaluated. In certain embodiments, likeliness to respond or time course is measured with respect to overall survival rate, time to progression and/or using the RECIST criteria.

It should be understood that the foregoing relates to certain embodiments of the invention and that numerous changes may be made therein without departing from the scope of the invention. The invention is further illustrated by the following examples, which are not to be construed in any way as imposing limitations upon the scope thereof. On the contrary, it is to be clearly understood that resort may be had to various other embodiments, modifications, and equivalents thereof, which, after reading the description herein may suggest themselves to those skilled in the art without departing from the spirit of the present invention and/or the scope the appended claims.

### EXAMPLES

The present invention may be better understood by reference to the following non-limiting examples.

### Example 1

### Tissue Samples

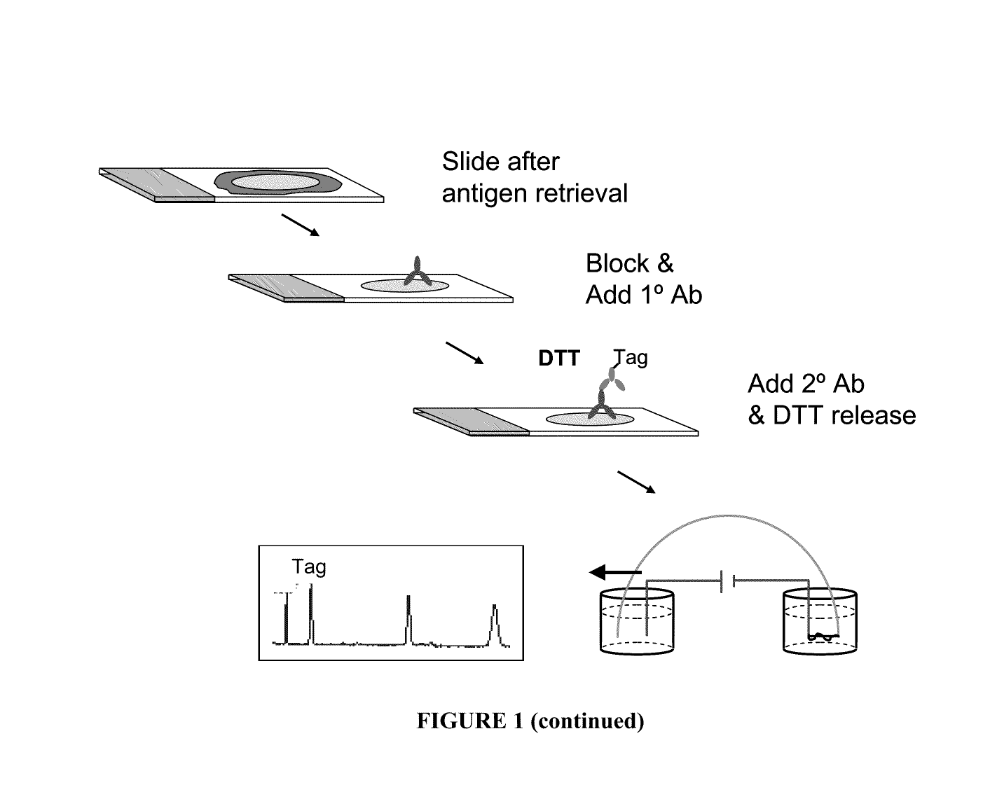

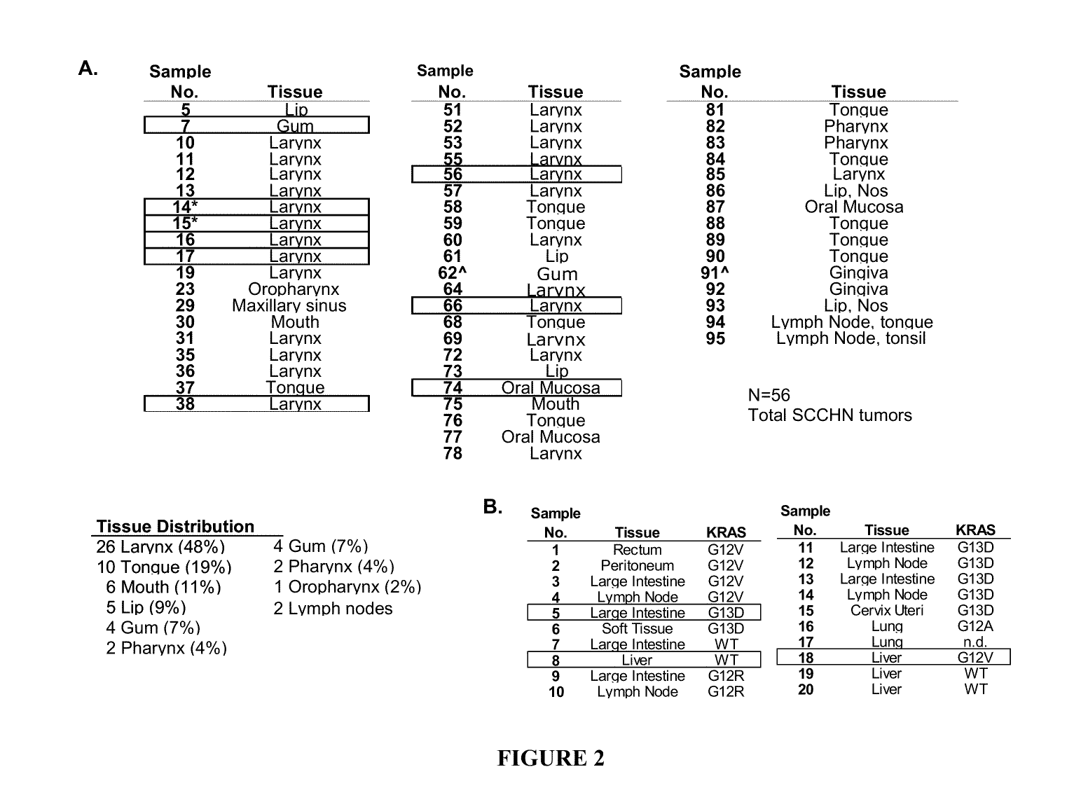

Fifty-six SCCHN tumor samples were purchased from Asterand, Inc. (Detroit, Mich.) or provided by Amgen, Inc. (Thousand Oaks, Calif.). FIG. 2A is a table showing the tissue source of each SCCHN tumor sample, as well as their percentage tissue distribution. Other head and neck cancers as described in Example 8 were also obtained from Asterand, Inc.

Twenty colorectal cancer samples were obtained from Asterand, Inc. FIG. 2 is a table identifying the source and the KRAS mutation status of each of the CRC samples. Samples identified by the experiments described below as having highly activated HER1 are boxed.

KRAS encodes GTPase KRas, which is an early player in many signal transduction pathways, particularly the HER1 signaling pathway. KRAS is a proto-oncogene in which single amino acid substitutions, and in particular single nucleotide substitutions, are activating mutations. The transforming GTPase KRAS protein is implicated in various malignancies, including lung adenocarcinoma, mucinous adenoma, ductal carcinoma of the pancreas and colorectal carcinoma. Thus, KRAS is frequently assessed for mutations in subjects with these cancers.

### Example 2

### Fixation, Processing and Paraffin Embedding

All tissues (0.3-1.0 gm) were fixed or snap-frozen within 15-30 minutes of excision. All tissues were fixed identically in neutral-buffered formalin (10% NBF) as dictated by the vendor's Standard Operating Procedure, which is consistent with the ASCO/CAP guidelines for preparation of breast tumor tissue for HER2 testing and other methods for HER1/EGFR fixation (i.e., 10% NBF for approximately 24 hrs at 4° C.). An independent study has shown preservation of phosphorylated HER expressed in xenograft tumors collected and processed in a similar manner (Mukherjee et al, 2011).

All cell-lines were maintained at 37° C. and 5% CO₂ in Dulbecco's modified Eagle medium (DMEM): F12 (50:50), 10% FBS, 1% PSQ (10% fetal bovine serum, 1% penicillin-streptomycin) and 2 mM L-glutamine. Cells were grown to near confluence on at least ten 150-mm culture plates for each cell line. After removal of medium, the cells were washed once with cold 1×PBS and 15 mL of 10% NBF (neutral buffered formalin) was added to each plate. Cells were fixed over night (>16 hrs) at 4° C. After removal of the fixative solution, the cells were harvested by scraping with residual fixative solution and centrifuged at 3200×g for 15 min. The cell pellet was transferred to a rubber O-ring, wrapped with filter paper and placed in a processing cassette. An automatic Tissue-Tek processor was used for dehydration and paraffin infusion processing. Briefly, cell pellet was exposed to increasing concentrations of alcohol, Clear-Rite clearing agent (xylene substitute) and paraffin. After processing, the cell pellet was embedded in a block using a paraffin embedding station. All solvents used for cell pellet processing were obtained from Richard-Allen Scientific.

### Example 3

### Microtomy

Sections of 5 μm in thickness were sliced with a microtome (LEICA) and placed on positively charged glass slides (VWR) with serial number labeled. Slides were air-dried for 30 minutes and then baked in a heated oven set at 60° C. for 1 hour. All sample slides were stored at 4° C. for future assays.

### Example 4

### Immunohistochemistry

Immunohistochemistry for HER1 was performed on Ventana Discovery XT system according to manufacturer's instructions. Primary antibody against HER1 was purchased from Invitrogen (cat #: 28-0005).

### Example 5

### Antibody Conjugate Reagents

Monoclonal antibodies against the extracellular domain of HER1, intracellular domain of HER2, intracellular domain of HER3, intracellular domain of HER1, phosphorylated tyrosine 1173 of HER1, and tyrosine phosphorylated HER1 were used. Primary antibody against HER1, HER2, HER3, and other reagents were purchased from Invitrogen (HER1 cat. #: 28-005, HER2 cat. #: 28-0003Z); Labvision (HER2 cat. #: MS-325 and MS-599, HER3 cat. #: MS-310); Cell Signaling (HER1 cat. #: 4267, phosphoHER1pY1173 cat. #: 4407); Leica/Novocastra (HER1 cat. #: NCL-L-EGFR); Millipore (phosphoYpan cat. #: 05-321); and Southern Biotech (Goat anti-Mouse IgG1 cat. #: 1070-01, Goat F(ab′)2 anti-Mouse IgG1 cat. #: 1072-01, Goat anti-Mouse IgG2a cat. #: 1080-01). Monoclonal antibodies against HER3 and c-MET were raised.

VeraTag® reporter molecules (Pro11 and Pro125) and streptavidin-conjugated methylene blue (“molecular scissors”) were synthesized and purified according to protocols described, for example, above and in U.S. Pat. Nos. 7,105,308 and 7,255,999, which are incorporated by reference in their entirety herein. VeraTag® reporter molecule-conjugated antibody and biotin-conjugated antibody were made using sulfo-NHS-LC-LC-biotin (Pierce) as linker according to manufacturer's protocol and conjugation products purified by HPLC (Agilent). In certain experiments, the VeraTag® reporter molecule or biotin was conjugated to a primary antibody. Depending on the assay format, the VeraTag® reporter molecule or biotin was conjugated to a secondary antibody that binds to the primary antibody. In the experiments described below, VeraTag® reporter molecule Pro125 was used to in assays to measure HER1 total, while VeraTag® reporter molecule Pro11 was used in assays to measure HER1-HER1 homodimers, HER1pY1173, HER1pYPan, HER2, HER3, HER1-HER2 heterodimers, and c-MET.

### Example 6

### VeraTag® Assay

Various VeraTag® assay formats are shown in FIG. 1. The assay format can be modified to use formalin-fixed paraffin-embedded (FFPE) tissue samples (Panel A) or tissue lysate samples (Panel B). In addition, the VeraTag® reporter molecules may be cleaved from antibodies to which they are bound using either a light release format (Panels A and B) or using a reducing format, e.g., using DTT (Panel C). In the light release format, diffusing reactive singlet oxygen cleaves the covalent linker between a VeraTag® reporter molecule and a HER1 antibody, for example, in response to photo-induction of the cleavage-inducing agent by light (“hv”). In the reducing format, a reducing agent such as, e.g., DTT, may be used to cleave the covalent linker between a VeraTag® reporter molecule and a HER1 antibody. Following cleavage, capillary electrophoretic (CE) separation may be performed to separate out the cleaved VeraTag® reporter molecules (tags), which can be visualized in an electropherogram. The x-axis of the resulting electropherogram shows the time at which the cleaved VeraTag® reporter molecule eluted from the capillary (i.e., based on electrophoretic mobility), and the fluorescence intensity is shown on the y-axis. Where the VeraTag® assay is formatted to detect the amount or presence of more that one protein target, target specific antibodies may be conjugated to VeraTag® reporter molecules having different electrophoretic mobilities so that each tag may be identified and quantified on the electropherogram.

The following method describes a general VeraTag® light-release assay that can be used to measure biomarker levels in a biological sample. The described method is particularly used for measurement of total biomarker levels (e.g., HER2 total).

Deparaffinization and antigen retrieval was generally performed as in (Sperinde et al, 2010, Clin. Cancer Res. 16(16):4226-4235; Shi, Y., et al., 2009, Diagn. Mol. Pathol. 18(1):11-21). FFPE samples were deparaffinized/rehydrated using a series of solvents. Briefly, slides were sequentially soaked in xylene (2×, 5 min), 100% ethanol (2×, 5 min), 70% ethanol (2×, 5 min) and deionized water (2×, 5 min). Heat-induced epitope retrieval of the rehydrated samples was performed in a dish containing 250 mL of 1× citrate buffer (pH 6.0) (Lab Vision) using microwave oven (Spacemaker II, GE): 3 min at power 10 followed by 10 min at power 3. After being cooled down for 20 min at room temperature, the slides were rinsed once with deionized water. A hydrophobic circle was drawn around the section on the slide using a hydrophobic pen (Zymed) to retain reagents on slides. The samples were then blocked for 1 hour with blocking buffer that contains 1% mouse serum, 1.5% BSA and a cocktail of protease and phosphatase inhibitors (Roche) in 1× PBS.

After removal of the blocking buffer with aspiration, a mixture of VeraTag® reporter molecule- and biotin-conjugated antibodies prepared in blocking buffer was added, and binding reactions were incubated overnight in a humidified chamber at 4° C. with shaking. The antibody mix was aspirated and samples were washed with buffer containing 0.25% Triton X-100 in 1×PBS, and streptavidin-conjugated methylene blue at a concentration of 2.5 μg/mL in 1×PBS was added. The concentrations of the antibody and streptavidin-photosensitizer conjugates were all optimized based on signal specificity and assay dynamic range using both cell line and breast tissue samples. After 1 hour incubation at room temperature, the streptavidin-methylene blue reagent was aspirated and the samples were washed in wash buffer once followed by 3 changes of deionized water. Illumination buffer containing 3 pM fluorescein and two CE internal markers (MF and ML) in 0.01×PBS was added on sample sections. The bound VeraTag® reporter molecule was released at ˜4° C. by photo-activated cleavage using an in-house LED array illuminator equipped with an electronic ice cube/chiller block (Torrey Pine Scientific). The CE sample containing the released VeraTag® reporter molecules was collected from above the tissue section on the slides and the released VeraTag® reporter molecules in the CE samples were separated and detected on an ABI3100 CE instrument (22-cm capillary array) (Applied Biosystems) under CE injection condition of 6 kV and 50 sec at 30° C.

The reducing (DTT) release assay format is similar but with the following general differences. After removal of the Blocking Buffer with aspiration, a solution containing the biomarker-specific antibody (e.g., HER1 antibody) in Blocking Buffer was added to the slides and left at 4° C. overnight in a humidified chamber with gentle shaking. The antibody solution was aspirated and samples were washed with PBS containing 0.25% TritonX-100 for 5 minutes then PBS alone for 5 minutes. Following aspiration, secondary antibody labeled with a VeraTag® reporter molecule in Blocking Buffer was added. The secondary antibody was allowed to incubate at room temperature for 1.5 hours in a humidified chamber. The slides were next rinsed with deionized water followed by PBS containing 0.25% Triton X-100 for 5 minutes. Slides were then loaded onto racks and submerged in deionized water 6 times. Following centrifugation of the slides, 100 μL Capture Buffer containing 1.0 mM dithiothreitol (DTT), 3 μM fluorescein and two CE internal markers (MF and ML) in 0.01×PBS was added on sample sections. Slides were incubated in a humidified chamber for 2 hours to allow for the release of the VeraTag® reporter molecule. Capture Buffer from each slide was transferred to a CE 96-well plate then diluted appropriately (generally 10-fold) in Capture Buffer not containing DTT. The released VeraTag® reporter molecules in the CE samples were separated and detected on a ABI3100 CE instrument (22-cm capillary array, Applied Biosystems) under the CE injection condition of 6 kV and 50 sec.

Other biomarkers as described herein may be detected using a VeraTag® assay similar to that as described for HER2 but using biomarker specific antibodies. These assays are identified in Table 1 below. Each of these VeraTag® assays used a blocking buffer of 10% goat serum (Sigma), 1 mg/ml hIgG (Sigma), 1.5% BSA and a cocktail of protease and phosphatase inhibitors (Roche) in 1× PBS. The concentrations of VeraTag® reporter molecule- and biotin-conjugated antibodies used for these assays are also indicated in Table 1.

TABLE 1
VeraTag ® Assay Formats

| Biomarker | Assay Conditions |
|---|---|
| HER1 Total | Release Method: DTT release method |
| | Antibody Format: single HER1 antibody (primary |
| | antibody) and single secondary antibody |
| | Antibody Concentrations: 0.2 μg/mL primary antibody |
| | and 0.4 μg/mL secondary antibody conjugated to |
| | a VeraTag ® reporter molecule |
| | Heat Retrieval Buffer: Diva Decloaker buffer, pH 6.2 |
| | (Biocare Medical) |
| HER2 Total | Release Method: light release method |
| | Antibody Format: two different HER2 antibodies |
| | Antibody Concentrations: both antibodies used at 4 μg/mL |
| | Heat Retrieval Buffer: Diva Decloaker buffer, pH 6.2 |
| HER3 Total | Release Method: light release method |
| | Antibody Format: two different HER3 antibodies |
| | Antibody Concentrations: both antibodies used at 2 μg/mL |
| | Heat Retrieval Buffer: DAKO Target Retrieval Solution, |
| | pH 9 (Dako Corporation) |
| HER1-HER1 | Release Method: light release method |
| homodimer | Antibody Format: single HER1 antibody - some antibody |
| | is conjugated with a VeraTag ® reporter molecule |
| | and some antibody is biotinylated |
| | Antibody Concentrations: both antibody conjugates |
| | used at 5 μg/mL |
| | Heat Retrieval Buffer: Diva Decloaker buffer, pH 6.2 |
| HER1pY1173 | Release Method: light release method |
| | Antibody Format: single HER1 antibody and single |
| | HER1pY1173 specific antibody |
| | Antibody Concentrations: both antibodies used at 2 μg/mL |
| | Heat Retrieval Buffer: Borg Decloaker buffer, |
| | pH 9.5 (Biocare Medical) |
| HER1pYpan | Release Method: light release method |
| | Antibody Format: single HER1 antibody and single pan- |
| | phosphotyrosine HER1 antibody |
| | Antibody Concentrations: both antibodies used at 2 μg/mL |
| | Heat Retrieval Buffer: Borg Decloaker buffer, |
| | pH 9.5 (Biocare Medical) |
| HER1-HER2 | Release Method: light release method |
| heterodimer | Antibody Format: single HER1 antibody, single HER2 |
| | antibody, first secondary antibody for HER1 antibody, |
| | second secondary antibody for HER2 antibody |
| | Antibody Concentration: HER1 primary antibody at |
| | 1:20.7 dilution and HER1 secondary conjugated |
| | to biotin at 2.5 μg/mL; HER2 primary antibody at |
| | 0.25 μg/mL and HER2 secondary conjugated to VeraTag ® |
| | reporter molecule at 2.5 μg/mL |
| | Heat Retrieval Buffer: Diva Decloaker buffer, pH 6.2 |
| c-MET | Release Method: light release method |
| | Antibody Format: two different c-MET antibodies - one |
| | antibody is conjugated with a VeraTag ® reporter molecule |
| | and one antibody is biotinylated |
| | Antibody Concentrations: 0.5 μg/mL for the |
| | VeraTag ® reporter molecule-conjugated antibody; |
| | 1 μg/mL for the biotin-conjugated antibody |
| | Heat Retrieval Buffer: Diva Decloaker buffer, pH 6.2 |

### Example 7

### VeraTag® Assay Batch Consistency

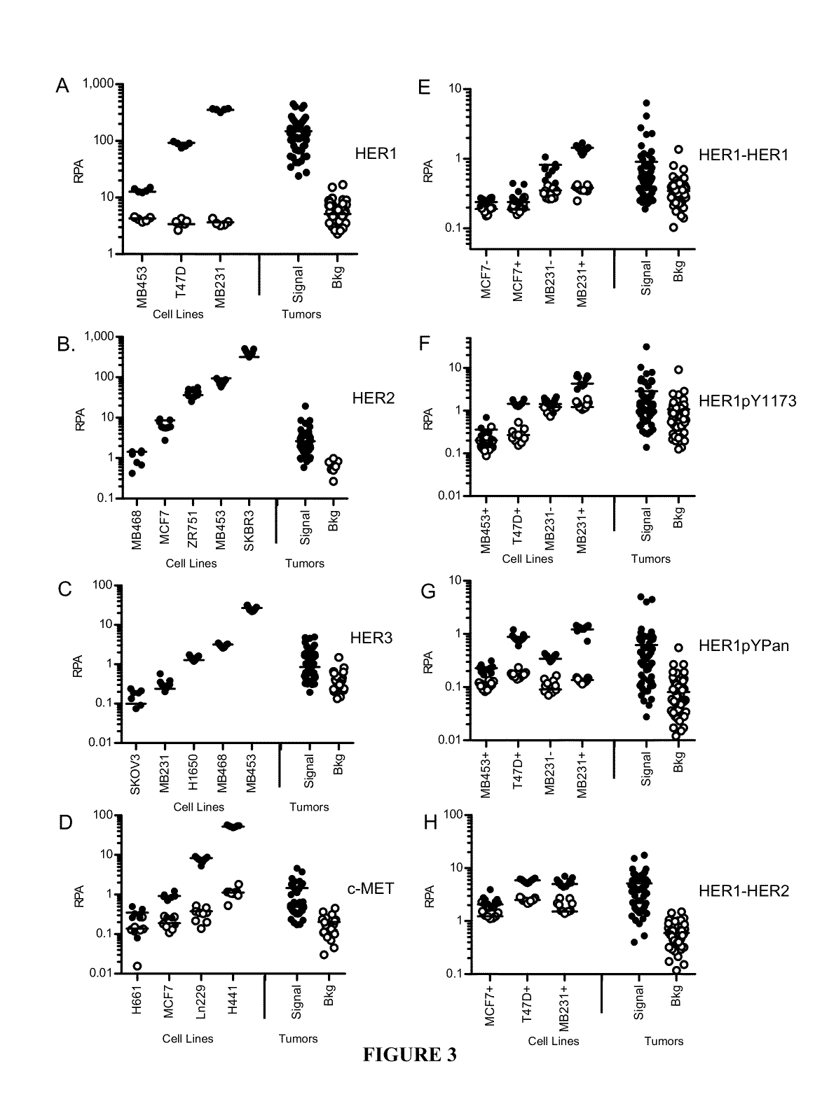

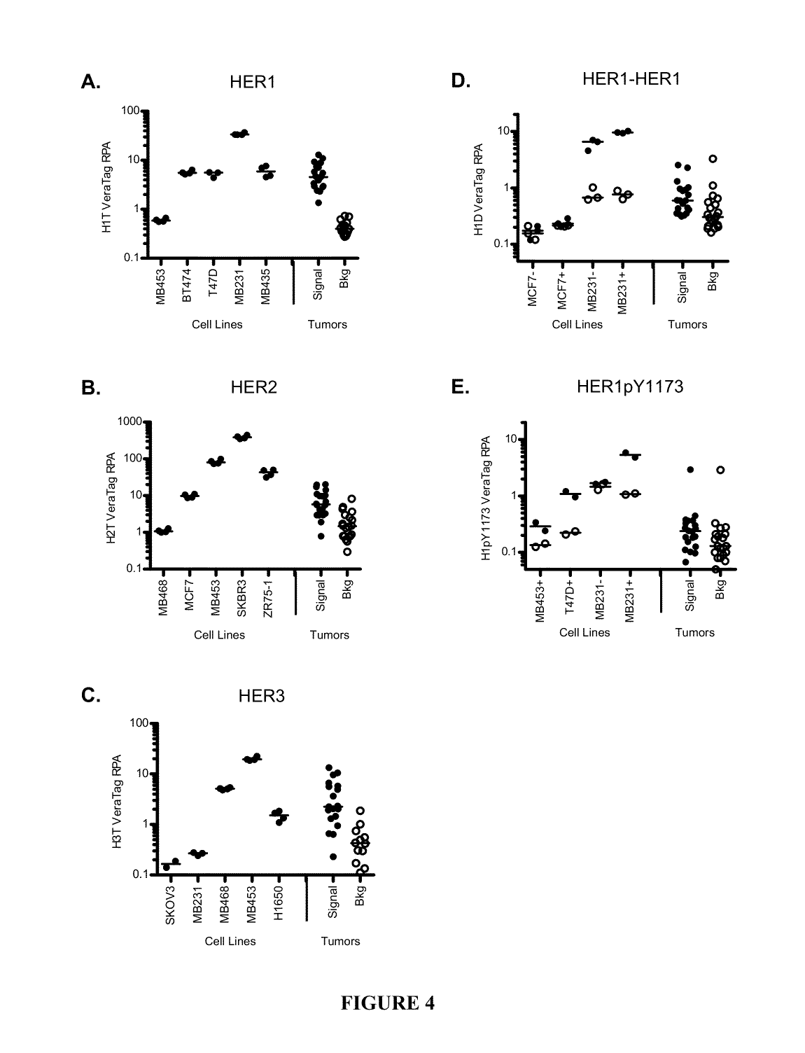

Due to the number of tumor samples assessed, VeraTag® assays were run for batches of tumor samples. The cell lines controls were also assessed for each batch to normalize measurements between batches. Scatter plots of biomarker measurements of the SCCHN and CRC samples are show in FIGS. 3 and 4, respectively. The fluorescence intensity for each sample is shown on the y-axis, measured in relative peak area (RPA). Control cell line measurements are on the left side of each graph and tumor sample measurements are on the right side of each graph. The solid dots indicate signal, and the open dots represent background.

Panels A, B, C, and D of FIG. 3 show results from assays measuring levels of total HER1, HER2, HER3, or c-MET, respectively. Panels E, F, G, and H of FIG. 3 show results from assays measuring activated HER1 (i.e., HER1-HER1 homodimers, HER1 phosphorylated at tyrosine 1173, pan-phosphorylated HER1, and HER1-HER2 heterodimers, respectively).

FIG. 4, Panels A-E, are graphs for the CRC samples. The biomarkers measured for each graph are: HER1, Panel A; HER2, Panel B; HER3, Panel C; HER1-HER1 homodimer, Panel D; HER1pY1173; Panel E.

The measurements for the cell line controls were found to be consistent from batch to batch, indicating the analytical reproducibility from batch to batch for the VeraTag® assay measurements.

### Example 8

### Biomarker Levels Differ Between SCCHN and Other Head and Neck Cancers

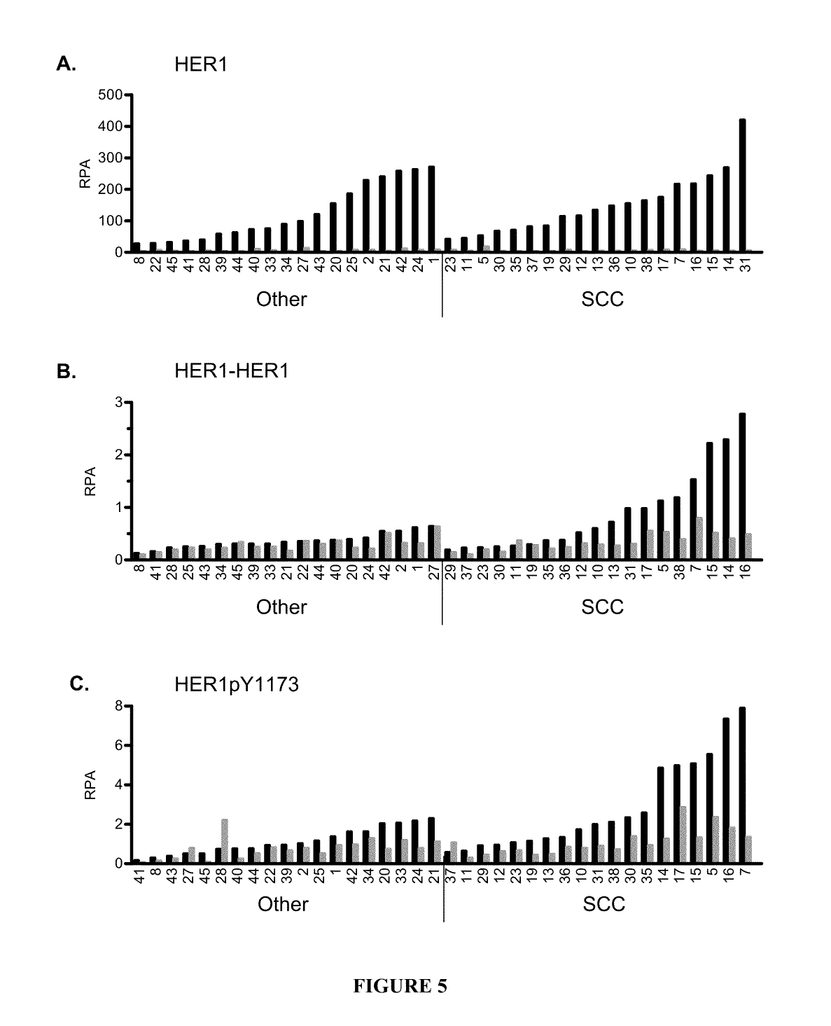

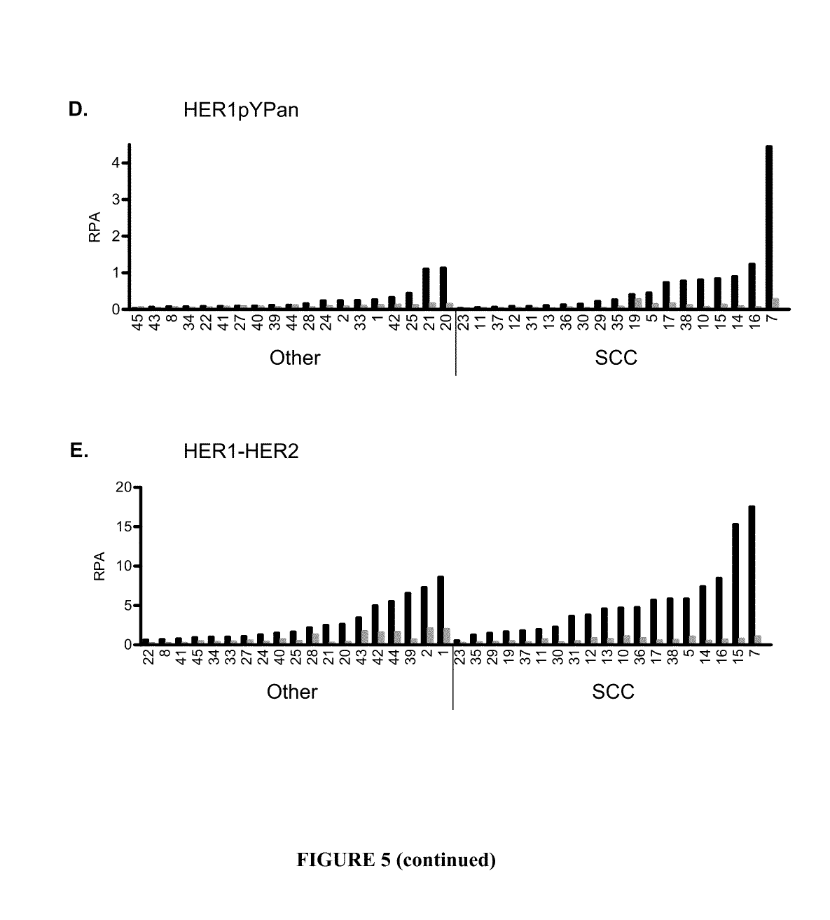

Graphs showing the amounts of different biomarkers in the SCCHN tumor samples and other head and neck tumors are shown in FIG. 5. The biomarkers assessed were: HER1 total (HER1), HER1-HER1 homodimers (HER1-HER1), HER1 phosphorylated at tyrosine 1173 (HER1pY1173), pan-phosphorylated HER1 (HER1pYPan), and HER1-HER2 heterodimers (HER1-HER2). See FIG. 5, Panels A-F, respectively. Biomarker levels were measured using VeraTag® assays. Biomarker levels were plotted using the box and whisker method. In each graph, the SCCHN samples are shown on the right side of the graph, and the other head and neck tumor samples are shown on the left side of the graph. Sample numbers are indicated on the x axis, and RPA/TA is shown on the y-axis. An unpaired t-test statistical analysis was performed to determine if the mean biomarker levels were different between the SCCHN and other head and neck cancers.

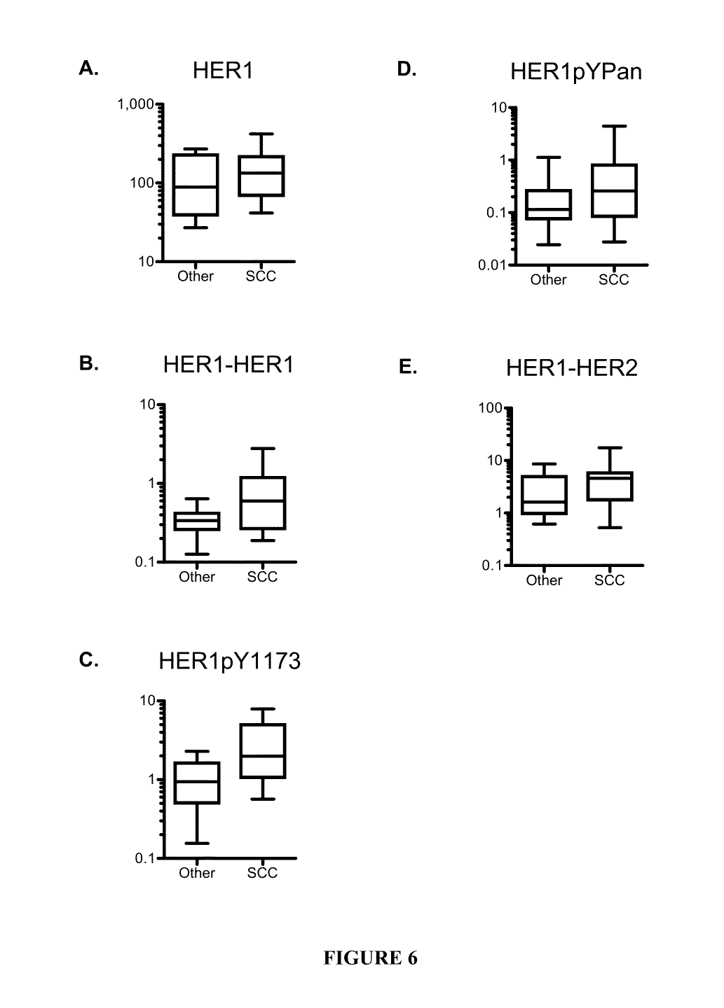

FIG. 6 shows the corresponding box and whisker plots of the biomarker level distribution when separating SCC tumors and Other tumors. The Spearman coefficient for each plot is identified. The amount of activated HER1 as measured by HER1-HER1 homodimers or Her1 phosphorylation (HER1pY1173) are statistically greater (p<0.05) in the SCC head and neck samples as compared to the “Other” head and neck tumors. However, total HER1 is not statistically greater in the SCC samples that the Other samples (p=0.4132) indicating that HER1 total alone is not sufficient to identify subjects having highly activated HER1 (FIG. 6A). The amount of activated HER1 as measured by HER1-HER2 heterodimers is nearly statistically greater (p=0.0569) in the SCC head and neck samples (FIG. 6E).

These data demonstrate that it is possible to distinguish SCCHN cancers from other head and neck tumors by measuring activated HER1 in the samples. The fact that SCCHN tumors had higher levels of HER1 total, HER1-HER1 homodimers and phosphorylated HER1 indicates that these cancers may be better targeted by HER1-activated drugs than other head and neck cancers.

### Example 9

### Cross Platform Comparisons

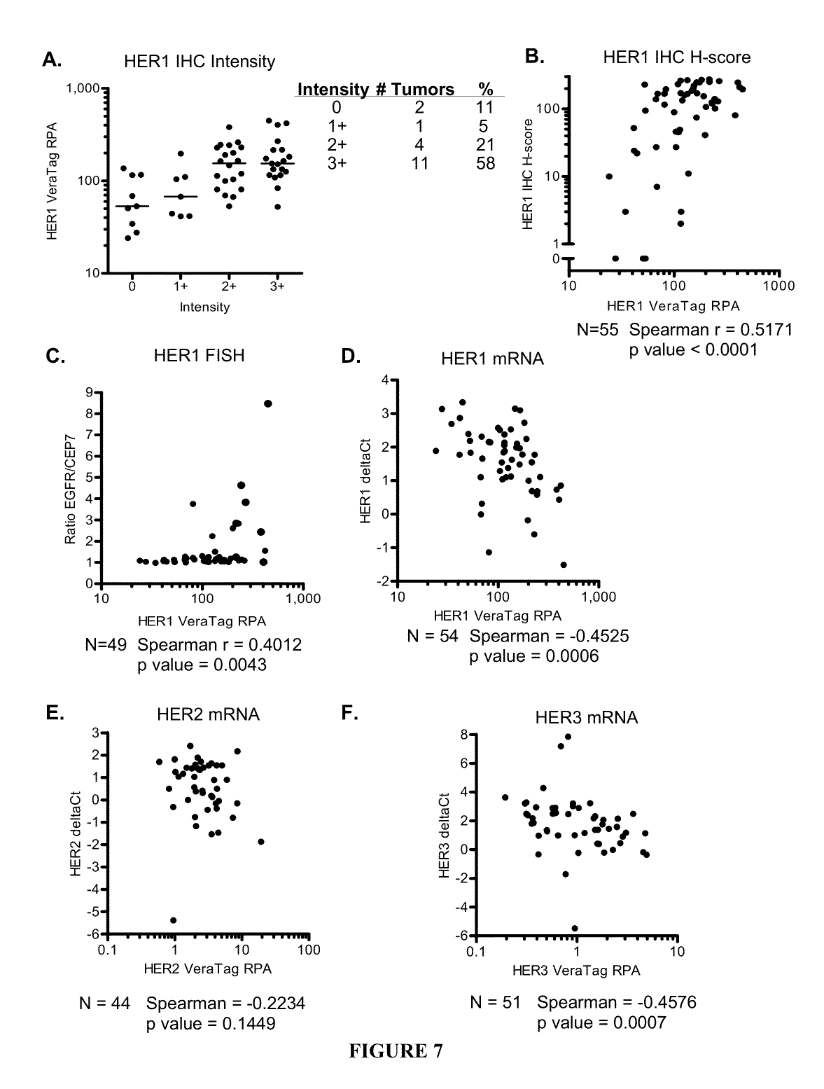

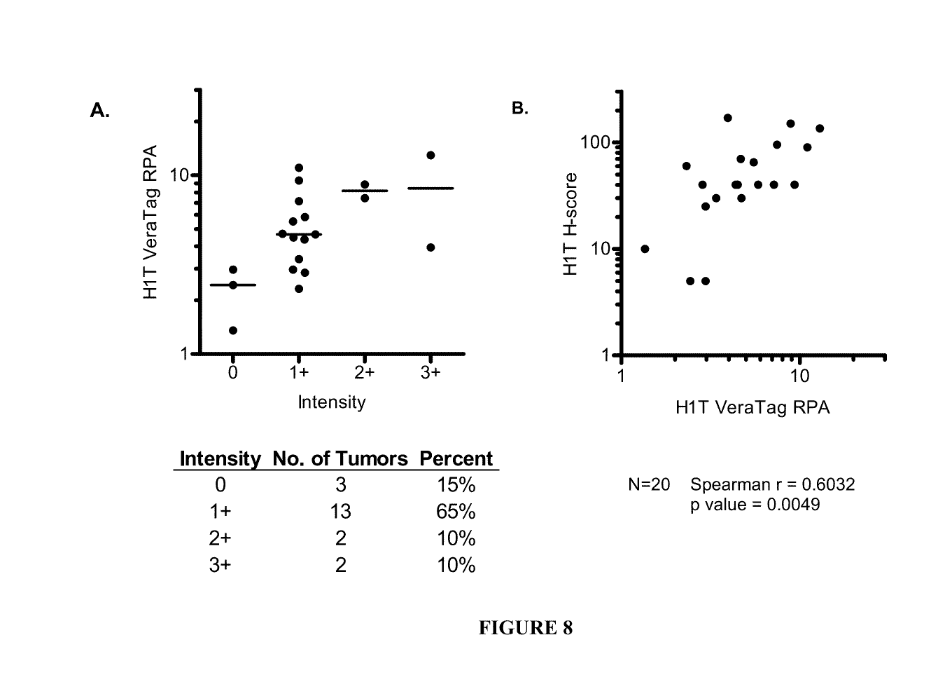

Experiments were conducted to compare VeraTag® assay measurements of biomarker levels to other standard methods of measurement gene expression. FIG. 7 shows the results of these experiments for the SCCHN samples and FIG. 8 shows the results for the CRC samples. Spearman's Rank Correlation test analysis was performed to identify a Spearman's rank correlation coefficient (Spearman) and p-value for each pairwise comparison.

FIG. 7, Panels A-F, are graphs comparing biomarker measurements across different platforms, including VeraTag® assays, quantitative PCR (mRNA), FISH, and IHC experiments from the same SCCHN tumors. Panels A and B compare VeraTag® assay results to IHC results. Panel C compares VeraTag® assay results to FISH assay results. Panels D, E, and F compare results of VeraTag® assays for HER1, HER2, and HER3, respectively, with the corresponding mRNA results measured as a deltaCt (change in cycle threshold), or the number of cycles necessary before the fluorescence reading surpasses the set baseline relative to an internal control.

FIG. 8, Panels A and B, are graphs comparing the results from the VeraTag® assays to the results from IHC experiments for the CRC tumor samples.

These data demonstrate that the measurement of protein levels using the VeraTag® assays significantly correlated with measurements of the biomarkers using other known standard measurement methods.

### Example 10

### Poor Stratification Based on Single Marker Analysis

The distribution of protein, mRNA, and/or gene copy levels were plotted to determine whether samples could be stratified based on single marker measurements alone. Horizontal lines represent the median measurement. Protein levels were measured by IHC or VeraTag assay. For IHC measurements, H-Score is a semi-quantitative measure of protein amount as assesed by IHC (intensity x area). mRNA levels were measured by quantitative PCR (qPCR), and gene copy levels were measured by FISH analysis.

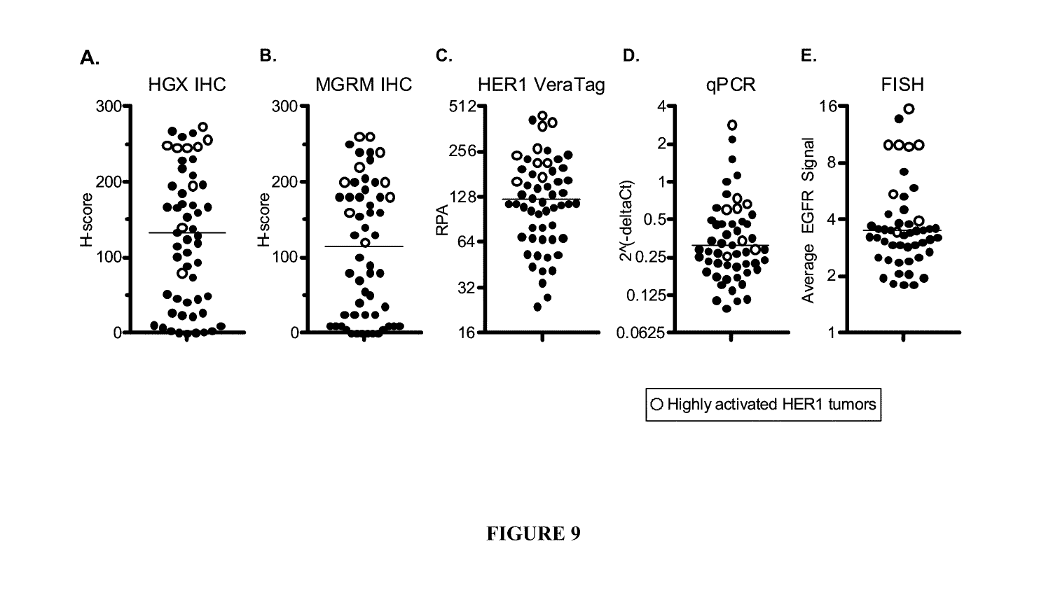

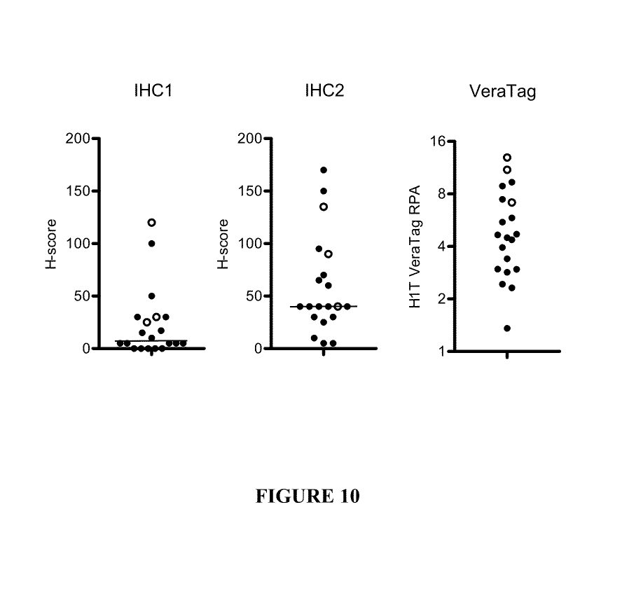

FIG. 9 shows scatter plot graphs of the distribution of HER1 measurements throughout the SCCHN tumors. The two IHC experiments in Panels A and B were conducted independently. FIG. 10 shows similar graphs plotting protein measurements based on IHC and VeraTag assay for the CRC tumors.

In each of the graphs, a relatively even distribution is observed for the samples with regards to the individual markers assessed. Thus, looking at protein, mRNA or gene copy levels alone does not stratify subjects to identify those that have the highest level of HER1 activation. In addition, combining the different HER1 expression measurements also does not stratify the highly activated HER1 tumors (e.g., combinations of FISH gene copy numbers with VeraTag®, IHC, and qPCR mRNA, or any other combinations of those measurements) (data not shown).

This data is consistent with Vermorken et al., 2008 and Vermorken et al., 2007, where IHC measurement of HER1 levels alone were unable to identify subjects having SCCHN that would be more likely to respond to treatment with cetuximab, a HER1-targeted agent.

### Example 11

### Spearman Correlation Coefficient Analysis

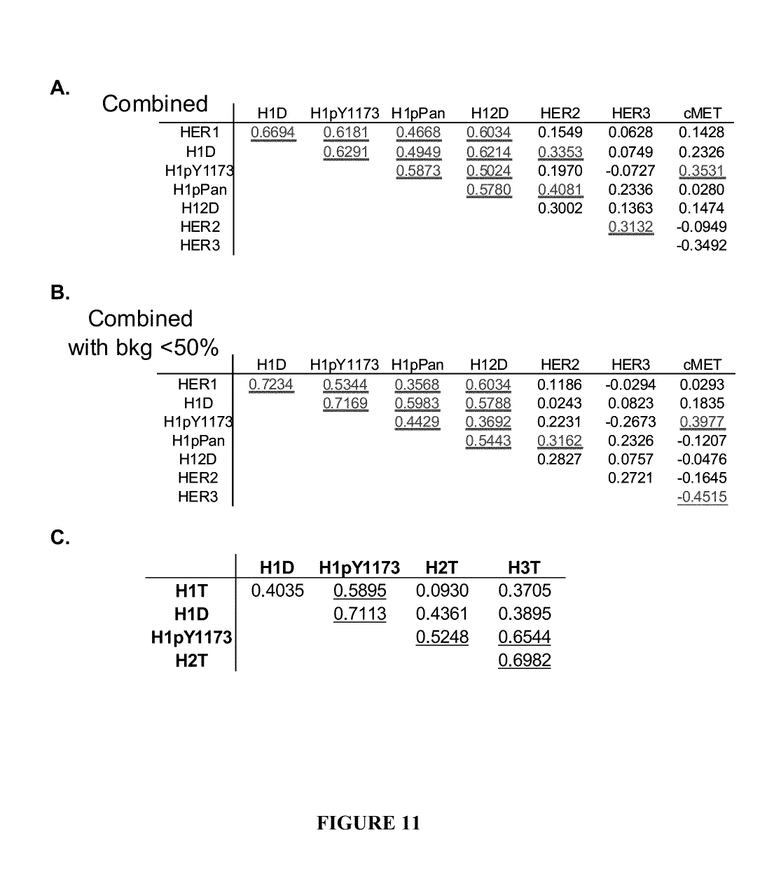

Statistical analysis of the biomarkers was conducted to determine if the levels of each biomarker was correlated to the level of any other biomarker in the tumor samples. Biomarker levels were measured using VeraTag® assays. Spearman's Rank Correlation test analysis was performed to identify a Spearman's rank correlation coefficient (Spearman) and p-value for each pairwise comparison of biomarkers. Tables showing the results of this statistical analysis are shown in FIG. 11 (Panels A and B: SCCHN samples; Panel C: CRC samples). The Spearman's rank correlation coefficients having significant p-values (p<0.05) are underlined.

A significant or near significant Spearman correlation coefficient was observed for several biomarkers. For example, HER1 total levels and HER1-HER1 homodimer levels were found to correlate in both SCCHN and CRC samples. HER1 total levels and HER1-HER1 homodimer levels were also found to significantly correlate with phosphorylated HER1 levels for the SCCHN samples (not measured in CRC samples). HER2 total levels were only found to have significant or near significant correlation to HER1 total levels and HER1-HER1 homodimer levels in the CRC samples.

### Example 12

### Heat Map Illustrating Correlation of Marker Levels

Biomarker levels were measured using VeraTag® assays and plotted onto heat maps to determine if a subset of samples for each cancer could be identified as having highly activated HER1. As noted previously, only approximately 13-16% of subjects with SCCHN respond to treatment with a HER-1 targeted therapy (cetuximab). See Vermorken et al., 2008, N. Engl. J. Med. 359, 116; Vermorken et al. 2007, J. Clin. Oncol. 25: 2171. As HER1-targeted therapies are more likely to be effective to treat cancers that are significantly driven by HER1 activation, identifying subjects with HER1-activated cancers may help to identify subjects who will respond better to HER1-targeted therapies.

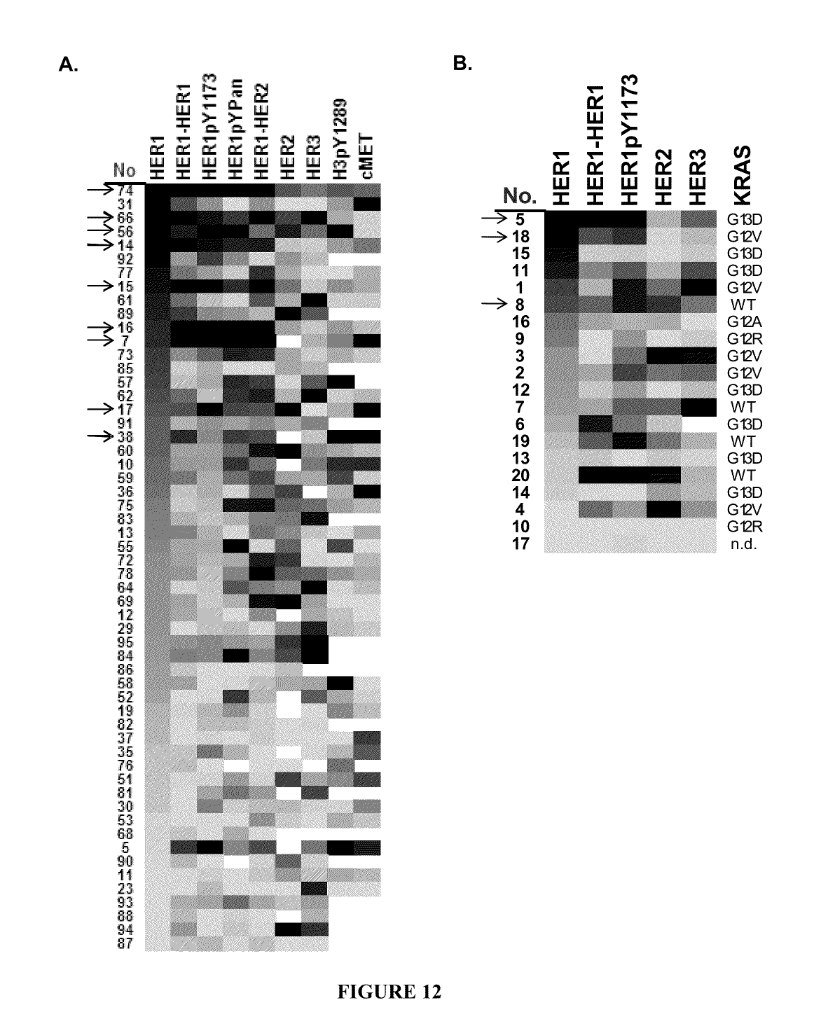

As shown in FIG. 12, biomarker levels for different biomarkers, as measured by VeraTag® assays, were plotted onto heat maps to identify expression patterns. Panel A is a heat map showing biomarker levels for each of the SCCHN tumor samples. Panel B is a heat map showing biomarker levels for the CRC tumor samples.

The sample number for each tumor sample is indicated to the left of each heat map, and the biomarker analyzed in the assay is shown at the top of each heat map. Samples that exhibited the highest expression (>90^{th} percentile) are shown in black or darker grey; and samples with low expression (<10^{th} percentile) are shown in light grey. Samples having the highest levels of activated HER1 are marked with an arrow. Samples were categorized as having the highest levels of activated HER1 based on evaluation of moderate to high HER1 total measurements combined with high levels of at least one of HER1-HER1 homodimers, HER1 phosphorylation, and HER1-HER2 heterodimers. For example, sample 38 in FIG. 12A has moderate levels of HER1 total but very high levels of HER1-HER1 homodimers. As dimerization is necessary for receptor activation, very high levels of dimerization are likely to indicate increased HER1 receptor activation. Another example is sample 17 in FIG. 12A, which had moderate levels of HER1 total but very high levels of HERlphosho1173 levels. As phosphorylation of HER1 at position 1173 activates HER1 signaling, high levels of this biomarker were also considered indicative of increased HER1 activation in a sample.

As shown in FIG. 12A, there were nine SCCHN samples identified that expressed the highest levels of activated HER1, representing ˜16% of the SCCHN tumors assessed. As shown in FIG. 12B, a similar proportion of samples was identified as containing activated HER1 in CRC samples (3/20=15%). Thus, measuring activated HER1 in the samples using the VeraTag® assays enabled identification of a subset of subjects that is consistent with the patient response rate of HER1-targeted therapy observed clinically (−13-16%;). Thus, this type of biomarker signature for activated HER1 may be used to identify subjects more likely to respond to HER1-targeted therapies.

### Example 13

### Hierarchical Cluster Analysis of SCCHN Samples

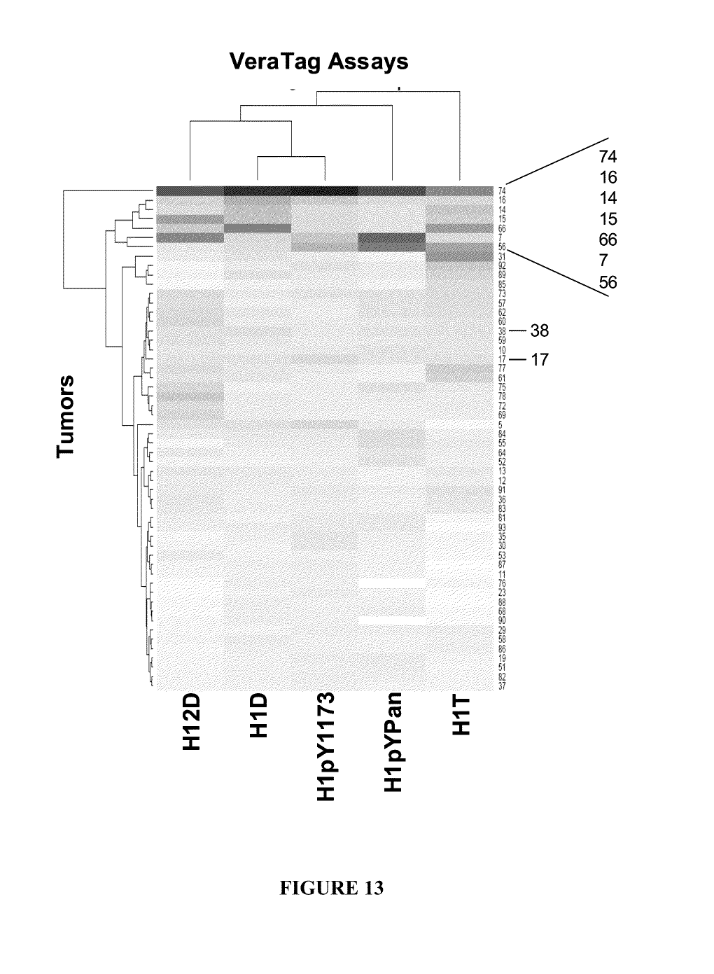

FIG. 13 shows a hierarchical cluster analysis of the SCCHN tumor samples by biomarker level as measured by VeraTag® assays. The sample numbers are shown on the right of the figure. The biomarkers analyzed are shown at the bottom of the figure: HER1-HER2 heterodimers (H12D); HER1-HER1 homodimers (H1D); HER1 phosphorylated at tyrosine 1173 (H1pY1173); pan-phosphorylated HER1 (H1pYPan); and total HER1 (H1T). The tumors expressing the highest levels of activated HER1 cluster into the top of the graph. The resulting dendogramidentified two closely related tumor clusters, one cluster having one tumor sample, and the other cluster having six tumor samples. These seven tumor samples were seven of the nine highly HER1 activated tumors identified in FIGS. 5 and 6, and represent 12.5% of the samples analyzed.

### Example 14

### Relative Activation Levels

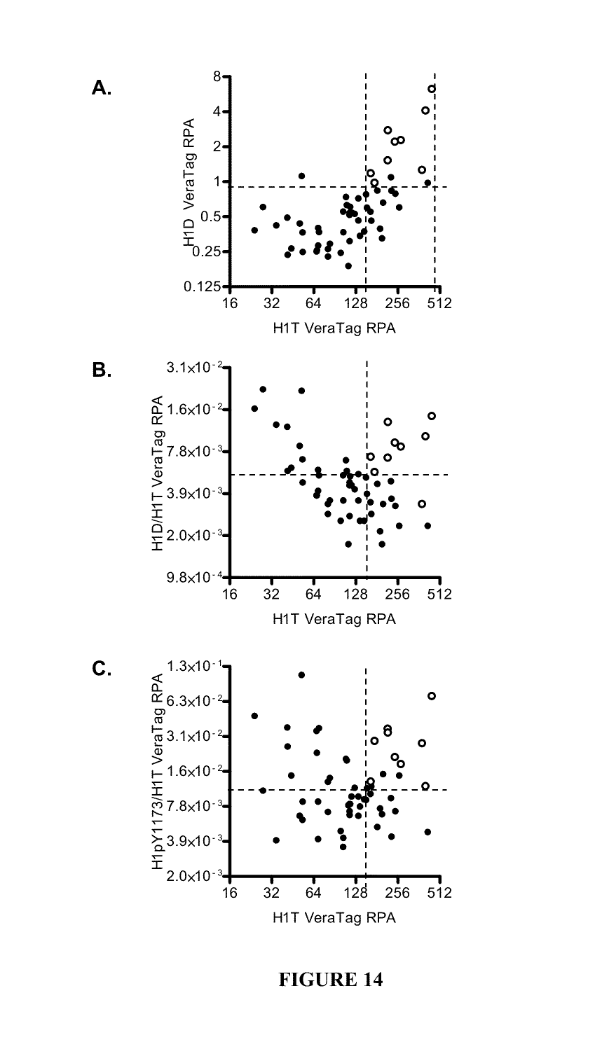

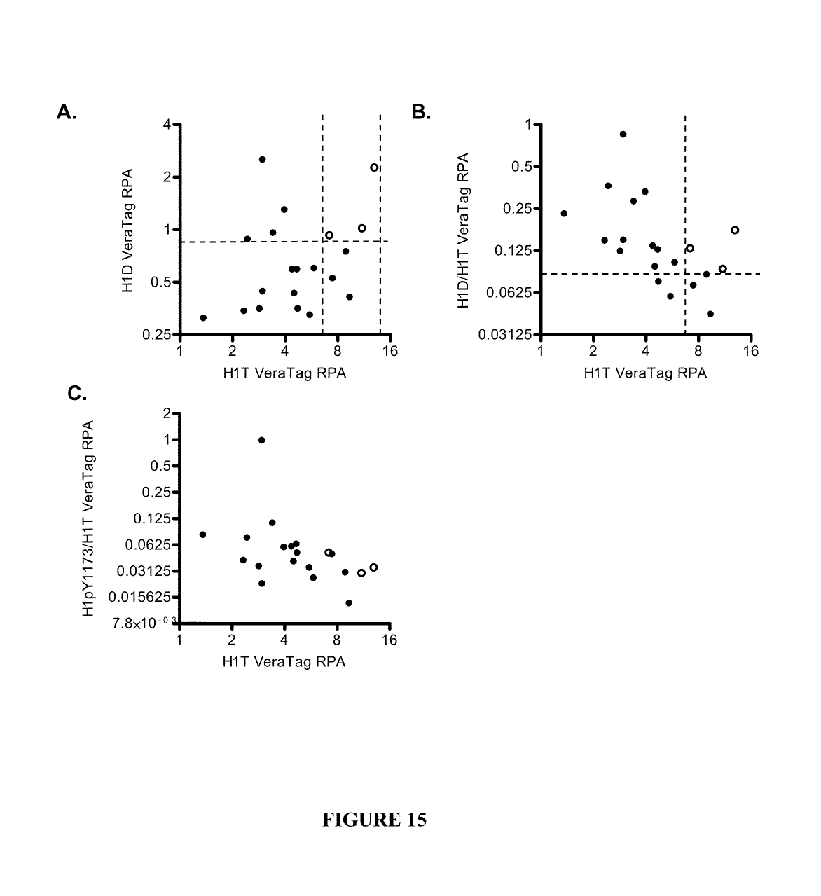

As it was determined that a single biomarker could not be used to stratify subject samples to identify those with highly activated HER1 that are more likely to be responsive to a HER1-targeted therapy, HER1 activation levels were plotted against biomarker levels to determine if a subset of subjects could be identified as having tumors with highly activated HER1. As noted above, subjects having highly activated HER1 are more likely to be responsive to HER1-targeted therapies. FIG. 14 shows plots based on SCCHN samples. FIGS. 15A-C shows plots based on CRC samples. Protein levels were each as measured using VeraTag® assays.

FIGS. 14A and 15A show results analyzing HER1-HER1 homodimers (H1D) versus HER1 total (H1T) expression levels. The tumors expressing the highest levels of activated HER1 fall into the top right quadrant. FIGS. 14B and 15B show results plotting the ratio of HER1-HER1 homodimers to HER1 total expression levels against HER1 total expression. The high HER1 total tumors expressing the highest levels of activated HER1 (as measured by HER1-HER1 homodimer expression) nearly all fall into the top right quadrant. FIGS. 14C and 15C show results analyzing the ratio of HER1 phosphorylated at tyrosine 1173 (H1pY1173) to HER1 total (H1T) versus HER1 total expression levels. The tumors expressing the highest levels of activated HER1 (as measured either by HER1-HER1 homodimerization or phosphorylation level (HER1pY1173)) nearly all fall into the top right quadrant. Of note, in each of these analyses, the patients who are within the top right quadrant are also those identified as having activated HER1 by heat map analysis.

In each of these analyses, by identifying subjects who had a combination of a high amount of HER1 total, a high amount of HER1-HER1 homodimer, a high ratio of HER1-HER1 homodimer to HER1 total, and/or high HER1 phosphorylation, it was possible stratify subjects to identify a subset of subject representing approximately 10-15% of the samples analyzed that could be classified as having tumors with high levels of HER1 activation.

All printed patents and publications referred to in this application are hereby incorporated herein in their entirety by this reference.

While the preferred embodiment of the invention has been illustrated and described, it will be appreciated that various changes can be made therein without departing from the spirit and scope of the invention.

1-23. (canceled)

24. A method of treating a subject with a cancer characterized as having high levels of activated HER1 comprising:
(a) identifying whether the subject's cancer has high levels of activated HER1 by (i) measuring the amount of total HER1 protein and at least one of HER1-HER1 homodimer, HER1-HER2 heterodimer, or phosphorylated HER1 in a biological sample from the subject, and (ii) determining if the tumor sample comprises elevated levels of total HER1 protein and at least one of HER1-HER1 homodimer, HER1-HER2 heterodimer, or phosphorylated HER1; and
(b) administering a HER1-targeted therapy to the subject.

25. The method of claim 24, wherein the cancer comprises at least one of squamous cell carcinoma of the head and neck (SCCHN), colorectal cancer, or lung cancer.

26. The method of claim 24, wherein the HER1-acting agent comprises at least one agent selected from the group consisting of cetuximab, gefitinib, erlotinib, lapatinib, panitumumab, zalatumumab, nimotuzumab, and matuzumab.

27-29. (canceled)

30. The method of claim 24, wherein the amount of phosphorylated HER1 in the biological sample is detected by using a HER1 phosphospecific or a HER1 pan antibody.

31. The method of claim 24, wherein the amount of phosphorylated HER1 in the biological sample is detected by using a phosphospecific antibody that binds HER1 protein that is phosphorylated at the tyrosine residue at position 1173 of HER1.

32. The method of claim 24, wherein the amount of activated HER1 in the biological sample is detected by determining the amount of at least two HER1 entities selected from the group consisting of HER1-HER1 homodimer, HER1-HER2 heterodimer, and phosphorylated HER1 that is present in the biological sample.

33. The method of claim 24, wherein the amount of activated HER1 is measured using an assay capable of measuring and/or quantifying an amount of protein-protein interactions in the biological sample.

34. The method of claim 24, further comprising detecting the level of at least one other biomarker selected from the group consisting of total HER2 protein, total HER3 protein, phosphorylated HER3, cMET.

35. The method of claim 34, further comprising determining the ratio of at least one of HER1-HER1 homodimer, HER1-HER2 heterodimer, or phosphorylated HER1 to total HER1 protein.

36. The method of claim 35, further comprising indicating that the subject has a cancer with high levels of activated HER1 if (i) the level of total HER1 in the sample is above the median level of total HER1 of a reference population and (ii) the ratio of at least one of HER1-HER1 homodimer to HER1 total, HER1-HER2 heterodimer to HER1 total, or phosphorylated HER1 to total HER1 in the sample is above the median ratio of at least one of HER1-HER1 homodimer to HER1 total, HER1-HER2 heterodimer to HER1 total, or phosphorylated HER1 to total HER1 in the reference population.

37. The method of claim 24, wherein measuring the amount of total HER1 protein in a biological sample from the subject comprises the steps of:
a) contacting the biological sample with a HER1 antibody composition;
b) contacting the HER1 antibody composition with a tagged binding composition, wherein the tagged binding composition comprises a molecular tag attached thereto via a cleavable linkage, and wherein the tagged binding composition specifically binds to the HER1 antibody composition;
c) cleaving the cleavable linker of the tagged binding composition, thereby releasing the molecular tag; and
d) quantitating the released molecular tag to determine the amount of HER1 protein in the biological sample.

38. The method of claim 24, wherein measuring the amount of total HER1 protein in a biological sample from the subject comprises the steps of:
a) contacting the biological sample with a first HER1 antibody composition that specifically binds to HER1 protein at a first binding site, wherein the first HER1 binding composition comprises a molecular tag attached thereto via a cleavable linkage;
b) contacting the biological sample with a cleaving probe that specifically binds to HER1 protein at a second binding site, wherein the cleaving probe cleaves the cleavable linkage of the HER1 antibody composition when within an effective proximity thereto;
c) cleaving the cleavable linker of the HER1 antibody composition, thereby releasing the molecular tag; and
d) quantitating the released molecular tag to determine the amount of HER1 protein in the biological sample.

39. The method of claim 24, wherein measuring the amount of HER1-HER1 homodimer in a biological sample from the subject comprises the steps of:
a) contacting the biological sample with a first HER1 antibody composition that specifically binds to HER1 protein at a first binding site, wherein the first HER1 binding composition comprises a molecular tag attached thereto via a cleavable linkage;
b) contacting the biological sample with a second HER1 antibody composition that also specifically binds to HER1 protein at the first binding site, wherein the second HER1 antibody composition comprises a cleavage-inducing moiety that cleaves the cleavable linkage of the HER1 antibody composition when within an effective proximity thereto;
c) cleaving the cleavable linker of the first HER1 antibody composition, thereby releasing the molecular tag; and
d) quantitating the released molecular tag to determine the amount of HER1-HER1 homodimer in the biological sample.

40. The method of claim 24, wherein measuring the amount of HER1-HER2 heterodimer in a biological sample from the subject comprises the steps of:
a) contacting the biological sample with an antibody composition comprising a molecular tag attached thereto via a cleavable linkage;
b) contacting the biological sample with a cleaving probe, wherein the cleaving probe cleaves the cleavable linkage of the antibody composition when within an effective proximity thereto;
c) cleaving the cleavable linker of the antibody binding composition, thereby releasing the molecular tag; and
d) quantitating the released molecular tag to determine the amount of HER1-HER2 heterodimer in the biological sample,
wherein the antibody composition binds specifically to HER1 and the cleaving probe binds specifically to HER2, or the antibody composition binds specifically to HER2 and the cleaving probe binds specifically to HER1.

41. The method of claim 24, wherein measuring the amount of HER1-HER2 heterodimer in a biological sample from the subject comprises the steps of:
a) contacting the biological sample with a HER1 antibody composition;
b) contacting the biological sample with a HER2 binding composition;
c) contacting the biological sample with a first binding composition that binds to either the HER1 antibody composition or the HER2 antibody composition, wherein the first binding composition comprises a molecular tag attached thereto via a cleavable linkage;
d) contacting the biological sample with a cleaving probe, wherein the cleaving probe cleaves the cleavable linkage of the binding composition when within an effective proximity thereto;
e) cleaving the cleavable linker of the antibody composition, thereby releasing the molecular tag; and
f) quantitating the released molecular tag to determine the amount of HER1-HER2 heterodimer in the biological sample,
wherein the cleaving probe binds specifically to HER2 if the antibody binding composition binds specifically to HER1, or the cleaving probe binds specifically to HER1 if the antibody binding composition binds specifically to HER2.

42. The method of claim 24, wherein measuring the amount of phosphorylated HER1 in a biological sample from the subject comprises the steps of:
a) contacting the biological sample with a first HER1 antibody composition that specifically binds to HER1 protein at a first binding site, wherein the first HER1 binding composition comprises a molecular tag attached thereto via a cleavable linkage;
b) contacting the biological tumor sample with a second HER1 antibody composition that specifically binds to HER1 protein at a second binding site, wherein the second HER1 antibody composition comprises a cleavage-inducing moiety that cleaves the cleavable linkage of the HER1 antibody composition when within an effective proximity thereto and wherein the second binding site comprises a HER1 phosphorylation site;
c) cleaving the cleavable linker of the first HER1 antibody composition, thereby releasing the molecular tag; and
d) quantitating the released molecular tag to determine the amount of phosphorylated HER1 in the biological sample.

43. A method for measuring the level of activated HER1 in a tumor, comprising:
(a) providing a sample from a tumor; and
(b) measuring the amount of total HER1 in the sample and the amount of at least one of HER1-HER1 homodimer, HER1-HER2 heterodimer, or phosphorylated HER1 in the sample.

44. The method of claim 43, further comprising determining the ratio of at least one of HER1-HER1 homodimer, HER1-HER2 heterodimer, or phosphorylated HER1 to total HER1 protein.

45. The method of claim 44, further comprising indicating that the tumor has a high level of activated HER1 if (i) the level of total HER1 in the sample is above the median level of total HER1 of a reference population and (ii) the ratio of at least one of HER1-HER1 homodimer to HER1 total, HER1-HER2 heterodimer to HER1 total, or phosphorylated HER1 to total HER1 in the sample is above the median ratio of at least one of HER1-HER1 homodimer to HER1 total, HER1-HER2 heterodimer to HER1 total, or phosphorylated HER1 to total HER1 in the reference population.

46. The method of claim 43, wherein the tumor comprises at least one of squamous cell carcinoma of the head and neck (SCCHN) cancer, colorectal cancer, or lung cancer.

47. The method of claim 43, wherein the amount of phosphorylated HER1 in the biological sample is detected by using a HER1 phosphospecific or a HER1 pan antibody.

48. The method of claim 43, wherein the amount of phosphorylated HER1 in the tumor is detected by using a phosphospecific antibody that binds HER1 protein that is phosphorylated at the tyrosine residue at position 1173 of HER1.

49. The method of claim 43, wherein the amount of activated HER1 in the tumor is detected by determining the amount of at least two HER1 entities selected from the group consisting of HER1-HER1 homodimer, HER1-HER2 heterodimer, and phosphorylated HER1 that is present in the tumor sample.

50. The method of claim 43, wherein the amount of activated HER1 is measured using an assay capable of measuring and/or quantifying an amount of protein-protein interactions in the tumor sample.

51. The method of claim 43, wherein measuring the amount of total HER1 protein in a tumor sample from the subject comprises the steps of:
a) contacting the tumor sample with a HER1 antibody composition;
b) contacting the HER1 antibody composition with a tagged binding composition, wherein the tagged binding composition comprises a molecular tag attached thereto via a cleavable linkage, and wherein the tagged binding composition specifically binds to the HER1 antibody composition;
c) cleaving the cleavable linker of the tagged binding composition, thereby releasing the molecular tag; and
d) quantitating the released molecular tag to determine the amount of HER1 protein in the tumor sample.

52. The method of claim 43, wherein measuring the amount of total HER1 protein in a tumor sample from the subject comprises the steps of:
a) contacting the tumor sample with a first HER1 antibody composition that specifically binds to HER1 protein at a first binding site, wherein the first HER1 binding composition comprises a molecular tag attached thereto via a cleavable linkage;
b) contacting the tumor sample with a cleaving probe that specifically binds to HER1 protein at a second binding site, wherein the cleaving probe cleaves the cleavable linkage of the HER1 antibody composition when within an effective proximity thereto;
c) cleaving the cleavable linker of the HER1 antibody composition, thereby releasing the molecular tag; and
d) quantitating the released molecular tag to determine the amount of HER1 protein in the tumor sample.

53. The method of claim 43, wherein measuring the amount of HER1-HER1 homodimer in a tumor sample from the subject comprises the steps of:
a) contacting the tumor sample with a first HER1 antibody composition that specifically binds to HER1 protein at a first binding site, wherein the first HER1 binding composition comprises a molecular tag attached thereto via a cleavable linkage;
b) contacting the tumor sample with a second HER1 antibody composition that also specifically binds to HER1 protein at the first binding site, wherein the second HER1 antibody composition comprises a cleavage-inducing moiety that cleaves the cleavable linkage of the HER1 antibody composition when within an effective proximity thereto;
c) cleaving the cleavable linker of the first HER1 antibody composition, thereby releasing the molecular tag; and
d) quantitating the released molecular tag to determine the amount of HER1-HER1 homodimer in the tumor sample.

54. The method of claim 43, wherein measuring the amount of HER1-HER2 heterodimer in a tumor sample from the subject comprises the steps of:
a) contacting the tumor sample with an antibody composition comprising a molecular tag attached thereto via a cleavable linkage;
b) contacting the tumor sample with a cleaving probe, wherein the cleaving probe cleaves the cleavable linkage of the antibody composition when within an effective proximity thereto;
c) cleaving the cleavable linker of the antibody composition, thereby releasing the molecular tag; and
d) quantitating the released molecular tag to determine the amount of HER1-HER2 heterodimer in the tumor sample,
wherein the antibody composition binds specifically to HER1 and the cleaving probe binds specifically to HER2, or the antibody composition binds specifically to HER2 and the cleaving probe binds specifically to HER1.

55. The method of claim 43, wherein measuring the amount of HER1-HER2 heterodimer in a biological sample from the subject comprises the steps of:
a) contacting the tumor sample with a HER1 antibody composition;
b) contacting the tumor sample with a HER2 binding composition;
c) contacting the tumor sample with a first binding composition that binds to either the HER1 antibody composition or the HER2 antibody composition, wherein the first binding composition comprises a molecular tag attached thereto via a cleavable linkage;
d) contacting the tumor sample with a cleaving probe, wherein the cleaving probe cleaves the cleavable linkage of the binding composition when within an effective proximity thereto;
e) cleaving the cleavable linker of the antibody composition, thereby releasing the molecular tag; and
f) quantitating the released molecular tag to determine the amount of HER1-HER2 heterodimer in the tumor sample,
wherein the cleaving probe binds specifically to HER2 if the antibody binding composition binds specifically to HER1, or the cleaving probe binds specifically to HER1 if the antibody binding composition binds specifically to HER2.

56. The method of claim 43, wherein measuring the amount of phosphorylated HER1 in a tumor sample from the subject comprises the steps of:
a) contacting the tumor sample with a first HER1 antibody composition that specifically binds to HER1 protein at a first binding site, wherein the first HER1 binding composition comprises a molecular tag attached thereto via a cleavable linkage;
b) contacting the tumor sample with a second HER1 antibody composition that specifically binds to HER1 protein at a second binding site, wherein the second HER1 antibody composition comprises a cleavage-inducing moiety that cleaves the cleavable linkage of the HER1 antibody composition when within an effective proximity thereto and wherein the second binding site comprises a HER1 phosphorylation site;
c) cleaving the cleavable linker of the first HER1 antibody composition, thereby releasing the molecular tag; and
d) quantitating the released molecular tag to determine the amount of phosphorylated HER1 in the tumor sample.

57. The method of claim 43, further comprising detecting the level of at least one other biomarker selected from the group consisting of total HER2 protein, total HER3 protein, phosphorylated HER3, cMET.
